(19) United States
(12) Patent Application Publication
(10) Pub. No.: US 2026/0351388 A1
(43) Pub. Date: Nov. 5, 2026

(54) SYSTEM AND METHOD FOR USE OF MACHINE LEARNING FOR TRAUMA TEAM ACTIVATION

(71) Applicant: UNIVERSITY OF ROCHESTER, Rochester, NY (US)

(72) Inventors: NICOLE A. WILSON, Rochester, NY (US); CATHERINE LIU, Rochester, NY (US)

(21) Appl. No.: 19/124,725

(22) PCT Filed: Feb. 5, 2024
(86) PCT No.: PCT/US2024/014379

§ 371 Date: Apr. 25, 2025

Related U.S. Application Data
(60) Provisional application No. 63/486,512, filed on Feb. 23, 2023.

(51) Int. Cl. G16H 40/20; G16H 10/60; G16H 50/70

(52) U.S. Cl. CPC G16H 40/20; G16H 10/60; G16H 50/70

(57) ABSTRACT

A method for assigning medical resources to a subject for treatment of traumatic injury, comprising the steps of (a) receiving, via a user interface of an application executing on one or more computer processors, a personal information profile; (b) assigning, via a machine learning model, a calculable value for any of a plurality of clinical conditions in the information profile; (c) determining, via a machine learning model, a total calculable value of said clinical conditions for said subject via the one or more computer processors, based on evaluation in real-time; (d) receiving a trauma activation notice determined by the relationship of the total calculable value to a trauma activation level defined by the machine learning model; (e) notifying, via said user interface, a medical professional providing medical care to said subject about the predicted assignment of medical resources for said subject.

This application claims priority from U.S. Provisional App. No. 63/486,512, filed Feb. 23, 2023, which is incorporated herein by reference.

This invention was made with government support under TR 001999 awarded by the National Institutes of Health. The government has certain rights in the invention.

### FIELD

This application relates generally to the field of machine learning and electronic healthcare and analysis and use in trauma care.

### BACKGROUND

Traumatic injuries accounted for 74.3% of deaths in children ages 1-18 years in 2020. While the development of multi-tiered trauma systems have improved survival, reduced morbidity, and decreased resource utilization, childhood morbidity and mortality from traumatic injury are increasing. These facts emphasize the importance of optimizing a comprehensive trauma system to match the needs of the injured patient with appropriate hospital resources and services.

The American College of Surgeons (ACS) Committee on Trauma (COT) Optimal Resources Document defines mandatory minimum criteria for trauma team activation (TTA) based on mechanism of injury, physiologic, and anatomic criteria. However, the variability in injury patterns and human physiologic responses to those injuries make it extremely challenging, if not impossible, to design TTA criteria with perfect sensitivity and specificity for hospital triage of trauma patients. There are no current electronic tools available to support critical clinical triage decisions upon arrival of an injured patient to a trauma center.

Therefore, a need exists for novel computer-implemented systems and methods for providing effective healthcare in the field of trauma care.

### SUMMARY

One aspect of the application is a method of training a machine learning model to predict the assignment of medical resources required to treat a patient for traumatic injury, the method comprising: (a) establishing a ground-truth, wherein the ground-truth corresponds to a standardized trauma activation level for a standardized patient with a standardized set of clinical conditions; (b) assigning a first trauma activation level for an individual patient identified with a plurality of clinical conditions selected from the standardized set of clinical conditions, wherein a machine learning model assigns the first trauma activation level with an algorithm containing a plurality of relative calculable values, wherein each relative calculable value is assigned to one or more clinical conditions in the standardized set of clinical conditions; (c) determining, by human medical review, for the individual patient which of the set of clinical conditions with which the individual patient presented; (d) determining, by human medical review, a second trauma activation level that matches the set of clinical conditions that the individual patient presented; (e) inputting into the machine learning model the second trauma activation level, determined by human medical review, based on the set of clinical conditions that the individual patient presented; (f) comparing the second trauma activation level, determined by human medical review, to the first trauma activation level, assigned by the machine learning model, based on the set of clinical conditions that the individual patient presented to the first trauma activation level, for an individual patient identified with a plurality of clinical conditions selected from the standardized set of clinical conditions; (g) determining, based on said comparison, whether the first trauma activation level assigned by the machine learning model corresponds to under-triage, over-triage, or an appropriate level of triage for the individual patient in a clinical environment; (h) comparing the second trauma activation level, determined by human medical review, based on the set of clinical conditions that the individual patient presented to the ground-truth; (i) determining, based on said comparison, whether the second trauma activation level corresponds to under-triage, over-triage, or an appropriate level of triage for the individual patient in a clinical environment; (j) adjusting one or more relative calculable values used by the machine learning model in assigning the first trauma activation level in response to the determination of whether the individual patient received under-triage, over-triage, or an appropriate level of triage for the individual patient in a clinical environment; and (k) repeating steps (b) to (j) with a plurality of patients, each identified with a plurality of clinical conditions selected from the set of clinical conditions until the machine learning model reaches an appropriate level of triage within desired parameters.

Another aspect of the present application relates to a method of training a machine learning model to predict the assignment of medical resources required to treat a patient for traumatic injury, the method comprising: (a) establishing a ground-truth, wherein the ground-truth corresponds to a standardized trauma activation level for a standardized patient with a standardized set of clinical conditions; (b) assigning a machine trauma activation level for an individual patient identified with a plurality of clinical conditions selected from the standardized set of clinical conditions, wherein a machine learning model assigns the first trauma activation level with an algorithm containing a plurality of relative calculable values, wherein each relative calculable value is assigned to one or more clinical conditions in the standardized set of clinical conditions; (c) comparing the machine trauma activation level to the ground-truth; (d) determining, based on said comparison, whether the machine trauma activation level corresponds to under-triage, over-triage, or an appropriate level of triage for the individual patient; (e) adjusting one or more relative calculable values used by the machine learning model in assigning the machine trauma activation level if the machine trauma activation level corresponds to under-triage or over-triage in step (d); and (f) repeating steps (b) to (e) with a plurality of patients, each identified with a plurality of clinical conditions selected from the set of clinical conditions until the machine learning model reaches an appropriate level of triage within desired parameters.

Another aspect of the present application relates to a method for assigning medical resources to a subject for treatment of traumatic injury, comprising the steps of (a) receiving, via a user interface of an application executing on one or more computer processors, a personal information profile, wherein said personal information profile comprises inputs for personal information for a plurality of types of information selected from the types of information comprising comorbidities, engineered features, prehospital interventions, injury mechanisms and numeric variables; (b) storing, via the one or more computer processors, said personal information of said subject in a database accessible by said application, and accessible by said subject via said user interface of said application; (c) assigning, via the one or more computer processors, a calculable value for any of a plurality of the types of information in said personal information stored in said database and a calculable value point matrix stored on a memory device accessible by the one or more computer processors, the calculable value point matrix being generated and continually updated by a machine learning module using a machine learning model trained by the method of the present application; (d) determining, via the one or more computer processors, a total calculable value of said types of information in said personal information for said subject via the one or more computer processors, based on evaluation in real-time, wherein when the total calculable value of said types of information in said personal information for said subject exceeds or falls below a pre-selected calculable value a predicted assignment of medical resources is generated; and (e) notifying, via said user interface, a medical professional providing medical care to said subject about the predicted assignment of medical resources for said subject.

Another aspect of the application is a system for assigning medical resources to a subject for treatment of traumatic injury in a subject, comprising: one or more computer processors; and one or more tangible computer readable media accessible by the one or more computer processors, wherein the one or more tangible computer readable media comprise instructions that, when executed by the one or more processors, cause the one or more processors to perform: (a) receiving, via a user interface of an application executing on one or more computer processors, a personal information profile, wherein said personal information profile comprises inputs for personal information for a plurality of types of information selected from the types of information comprising comorbidities, engineered features, prehospital interventions, injury mechanisms and numeric variables; (b) storing, via the one or more computer processors, said personal information of said subject in a database accessible by said application, and accessible by said subject via said user interface of said application; (c) assigning, via the one or more computer processors, a calculable value for any of a plurality of the types of information in said personal information stored in said database and a calculable value point matrix stored on a memory device accessible by the one or more computer processors, the calculable value point matrix being generated and continually updated by a machine learning module using a machine learning model trained by the method described herein; (d) determining, via the one or more computer processors, a total calculable value of said types of information in said personal information for said subject via the one or more computer processors, based on evaluation in real-time, wherein when the total calculable value of said types of information in said personal information for said subject exceeds or falls below a pre-selected calculable value a predicted assignment of medical resources is generated; and (e) notifying, via said user interface, a medical professional providing medical care to said subject about the predicted assignment of medical resources for said subject.

An aspect of the application is a tangible non-transitory computer readable storage medium, comprising instructions that, when executed by a computer processor, cause the processor to: (a) receiving, via a user interface of an application executing on one or more computer processors, a personal information profile, wherein said personal information profile comprises inputs for personal information for a plurality of types of information selected from the types of information comprising comorbidities, engineered features, prehospital interventions, injury mechanisms and numeric variables; (b) storing, via the one or more computer processors, said personal information of said subject in a database accessible by said application, and accessible by said subject via said user interface of said application; (c) assigning, via the one or more computer processors, a calculable value for any of a plurality of the types of information in said personal information stored in said database and a calculable value point matrix stored on a memory device accessible by the one or more computer processors, the calculable value point matrix being generated and continually updated by a machine learning module using a machine learning model trained by the method described herein; (d) determining, via the one or more computer processors, a total calculable value of said types of information in said personal information for said subject via the one or more computer processors, based on evaluation in real-time, wherein when the total calculable value of said types of information in said personal information for said subject exceeds or falls below a pre-selected calculable value a predicted assignment of medical resources is generated; and (e) notifying, via said user interface, a medical professional providing medical care to said subject about the predicted assignment of medical resources for said subject.

### BRIEF DESCRIPTION OF FIGURES

FIG. 1, Panel A shows a general overview of the steps in training a machine learning model for the methods and systems herein. Panel B shows a focused overview of the model training and assessment for the machine learning model.

FIG. 2 shows of how the feature weights relate to the output from the algorithm.

FIG. 3, Panel A shows feature importance when Cribari is the ground-truth. Panel B shows feature importance when NFTI is the ground-truth. Panel C shows feature importance when Cribari+NFTI is the ground-truth.

FIG. 4 shows proportion of patients under-triaged by each ground-truth.

FIG. 5 shows different types of algorithms that may be used in the methods and systems herein.

FIG. 6, Panel A shows undertriage rate and number of patients in dataset by injury mechanism. The bars represent the undertriage rate (left axis) and the line on the plot shows the total number of patients in the dataset with each mechanism of injury. Panel B shows rate of undertriage by ED staff for different patient arrival hours (bars, left y-axis) along with admission rate of patients for these times (line, right y-axis).

FIG. 7 shows Cribari ground truth trauma activation levels by time of patient admission.

FIG. 8 shows under and overtriage rates for patients of different age. Ages have been 0-1 normalized.

FIG. 9 shows comparison of undertriage rates for patients above and below the median age in our dataset of 47.57 years.

FIG. 10, Panel A shows distribution of activation ground truth variable across all data. Panel B shows distribution of activation ground truth variable within different variables.

FIG. 11 shows example output of LIM E explainability method.

FIG. 12 shows receiver operating characteristic (ROC) curves for each of the benchmarked machine learning models. ROC curves illustrate the discriminatory ability of a binary classifier by plotting the true positive rate against the false positive rate at various thresholds. The higher the area under the curve (AUC), the higher separability between the predictions. A random chance classifier achieves an AUC of 0.5, which is illustrated by the dashed black line. The highest performance was achieved by the support vector machine (red line), followed by logistic regression (blue line), random forest (purple), and ED staff (yellow).

FIG. 13 shows feature importance for the Support Vector Machine model. Engineered features were built from existing institutional triage criteria, other features were built from mechanism of injury, comorbidities, and pre-hospital interventions.

FIG. 14 shows distribution of full and partial trauma team activations, as determined by trained emergency department (ED) staff. (A) ED staff triggered a full activation in 230 (16.8%) patients and a partial activation in 1,136 patients (83.2%). (B) With the exception of gunshot wounds (GSW), full trauma team activations ranged from 9.1-18.5% for all mechanisms of injury. The distribution differed for gunshot wounds, with 50.4% triggering a full trauma team activation. Abbreviations: ED: Emergency Department; GSW: Gunshot Wound; MVC: Motor Vehicle Collision.

FIG. 15 shows (A) Proportion of patients predicted to require a full activation by each ground-truth. The Union (U) of the two sets Cribari+NFTI is shown outside of the colored circles The intersection (n) of Cribari+NFTI is shown. (B) Proportion of patients who received full activation and those prediction to require full activation. Proportion of patients that actually received a full activation (ED staff) and proportional overlap by those predicted by Cribari and NFTI. There was complete agreement in only 96 patients.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

### 1. DEFINITIONS

As used herein, the term “computer” refers to a machine, apparatus, or device that is capable of accepting and performing logic operations from software code. The term “application”, “software”, “software code” or “computer software” refers to any set of instructions operable to cause a computer to perform an operation. Software code may be operated on by a “rules engine” or processor. Thus, the methods and systems of the present invention may be performed by a computer or computing device having a processor based on instructions received by computer applications and software.

The term “electronic device” as used herein is a type of computer comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of electronic devices include: personal computers (PCs), workstations, laptops, tablet PCs including the iPad, cell phones including iOS phones made by Apple Inc., Android OS phones, Microsoft OS phones, Blackberry phones, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, wearable computers such as Apple Watch, other smartwatches, Fitbit, other wearable fitness trackers, Google Glasses, and the like.

The term “client device” as used herein is a type of computer or computing device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of client devices include: personal computers (PCs), workstations, laptops, tablet PCs including the iPad, cell phones including iOS phones made by Apple Inc., Android OS phones, Microsoft OS phones, Blackberry phones, Apple iPads, A nota digital pens, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, tablets, digital pens, wearable computers such as Apple Watch, other smartwatches, Fitbit, other wearable fitness trackers, Google Glasses, and the like.

The term “computer readable medium” as used herein refers to any medium that participates in providing instructions to the processor for execution. A computer readable medium may take many forms, including but not limited to, non-volatile media, volatile media, and transmission media. Non-volatile media includes, for example, optical, magnetic disks, and magneto-optical disks, such as the hard disk or the removable media drive. Volatile media includes dynamic memory, such as the main memory. Transmission media includes coaxial cables, copper wire and fiber optics, including the wires that make up the bus. Transmission media may also take the form of acoustic or light waves, such as those generated during radio wave and infrared data communications.

As used herein the term “data network” or “network” shall mean an infrastructure capable of connecting two or more computers such as client devices either using wires or wirelessly allowing them to transmit and receive data. Non-limiting examples of data networks may include the internet or wireless networks or (i.e. a “wireless network”) which may include Wifi and cellular networks. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, a Zigby network, or a voice-over-IP (VOIP) network.

As used herein, the term “database” shall generally mean a digital collection of data or information. The present invention uses novel methods and processes to store, link, and modify information such digital images and videos and user profile information. For the purposes of the present disclosure, a database may be stored on a remote server and accessed by a client device through the internet (i.e., the database is in the cloud) or alternatively in some embodiments the database may be stored on the client device or remote computer itself (i.e., local storage). A “data store” as used herein may contain or comprise a database (i.e. information and data from a database may be recorded into a medium on a data store).

As used herein, the term “comorbidities” refers to the simultaneous presence of two or more diseases or medical conditions in a patient. In certain embodiments, co-morbidities may include one or more of, Active Chemotherapy, Advanced Directive Limiting Care, Alcoholism, Alzheimers Disease, Anemia, Angina Pectoris, Anticoagulant Therapy, Ascites within 30 Days, Asthma, Attention Deficit Disorder/Attention Deficit Hyperactivity Disorder, Bleeding Disorder, Cancer, Cardiopulmonary Resuscitation (CPR), Cerebrovascular Accident (CVA), Chronic Obstructive Pulmonary Disease (COPD), Chronic Renal Failure, Cirrhosis, Concurrent or Existence of Metastasis, Congestive Heart Failure, Congenital Anomalies, Coronary Artery Diseases, Coumadin Therapy, Current Smoker, Currently Receiving Chemotherapy for Cancer, Chronic Demyelinating Disease, Chronic Drug Abuse, Crohn's Disease, CVA/Hemiparesis (Stroke with Residual), Dementia, Diabetes Mellitus, Dialysis (Excludes Transplant Patients), Disseminated Cancer, Documented History of Cirrhosis, Documented Prior History of Pulmonary Disease with Ongoing Active Treatment, Drug Use Disorder, DVT, Esophageal Varices, Functionally Dependent Health Status, Heart Disease, Hemophilia, History of Angina within Past 1 Month, History of Cardiac Surgery, History of Myocardial Infarction, History of Peripheral Vascular Disease (PV D), History of Psychiatric Disorders, HIV/AIDS, Hypertension, Insulin Dependent, Inflammatory Bowel Disease, Irritable Bowel Disease, Major Psychiatric Illness, Mental/Personality Disorder, Multiple Sclerosis, Myocardial Infarction (MI), Non-Insulin Dependent Diabetes Mellitus, Obesity, Pancreatitis, Parkinsons Disease, Peripheral Arterial Disease (PAD), Peripheral Vascular Disease (PVD), Pregnancy, Prehospital Cardiac Arrest with Resuscitative Efforts by Healthcare Provider, Prehospital CPR, Prematurity, Psychiatric Disorder, Pulmonary Embolus (PE), Renal Failure, Rheumatoid Arthritis, Routine Steroid Use, Seizures, Spinal Cord Injury, Steroid Use, Substance Abuse Disorder, Systemic Lupus Erythematous, Transplant, Ulcerative colitis, Undergoing Current Therapy, and Vascular Disease.

As used herein, the term “prehospital interventions” refers to medical treatments or interventions occurring before or during transportation (as of a trauma victim) to a hospital. In certain embodiments, pre-hospital interventions may include one or more of, Airway, Airway Cleared, Alternative Airway Device, Assisted Vent w/BVM, Bleeding Control, Chest Compression, Combitube, CPR Initiated by Crew, CPR in Progress, CPR Continued, Endotracheal Tube, EKG, EKG Monitor, Hemorrhage Control, Hemostatic Dressing, IO, IO fluids < or =500 cc, IO fluids 500-2000 cc, IO fluids > or =2000 cc, IO fluids unknown amount, IO fluids attempted, IV Fluids, IV Fluids <500 ml, IV Fluids 500-2000 ml, IV Fluids >2000 ml, IV Fluids amount unknown, IV fluids attempted, King Airway, Limb Immobilized, Medication Administered, Nasal ETT, Nasal Trumpet, Needle Decomp of Tension PTX, Needle Thoracostomy, Oral Airway, Oxygen, Oxygen by Cannula, Oxygen by M ask, Pelvic Binder, Pressure, Saline, SPINE, Spinal Immobilization, Suction, and Tourniquet.

As used herein, the term “mechanisms of injury” refers to the method by which damage (trauma) to skin, muscles, organs, and bones occurs. In certain embodiments, mechanisms of injury may include one or more of, Abuse, Aircraft, Airplane Crash, All Terrain Vehicle, Bite-Animal, human, Assault, Bicycle Crash, Boating accident, Broken Glass, Burn, Chainsaw, Child Abuse, Crush Injury, Dirt Bike, Diving, Drowning, Electrical Injury, Explosion, Fall, Fall-tree stand, Fall from snowboard, Farm/Heavy Equipment Incident, Fight/Brawl, Fireworks, Gunshot Wound, Hand and Table Saw, Hanging, Industrial Incident, Injured by Animal, Jet Ski, Jump, Motorcycle Crash, Motor Cross Accident, Motor Pedestrian Crash, Motor Vehicle Crash, Motorcycle Accident, Open wound/dehiscence, Pedestrian struck, Playground, Power lawnmower, Riding mower, Roller skates/blades, Scooter, Shooting-BB Gun, Skateboard, Skiing, Sledding or Tubing, Snow Mobile Crash, Sports-occurred while engaged in a sporting activity, Stab Wound, Struck by an object-non-motor vehicle related, Struck in Sports, Surgery, Trampoline, and Unknown.

As used herein, the term “calculable value” refers to a value that is assigned that is able to be expressed as an amount, quantity, or numerical value. In certain embodiments, the calculable value is a weight score assigned to a particular factor in the algorithm employed by the systems and methods described herein.

As used herein, the term “institutional triage criteria” refers to the criteria to determine whether a patient is classified as regarding full trauma triage or not. In certain embodiments, the institutional triage criteria may include: (Level 1) Confirmed blood pressure <90 mmHg at any time in adults; Age-specific hypotension in children; Gunshot wound to neck, torso, groin, buttock or junctional zones; Respiratory compromise or pre-arrival intubation; GCS <8 with mechanism attributed to trauma; Transfer patient receiving or has received blood transfusion; CPR in progress or history of CPR following trauma; Discretion of EM attending, triage nurse or communications nurse; (Level II) Gunshot wound to head; Gunshot wound to arm/leg proximal to elbow/knee; Stab wound to head, neck, torso, groin or junctional zone; Active bleeding requiring a tourniquet or wound packing; Suspected spinal injury with new motor or sensory loss; Traumatic amputation proximal to wrist or ankle; Two or more suspected proximal bone fractures: (to include suspected pelvic fractures, open fractures); Discretion of EM attending, triage nurse, or communications nurse; (Level III) Significant Head Injury (skull fracture or ICH) with torso or extremity trauma, not otherwise meeting level criteria; Multiple rib fractures, flail chest, not otherwise meeting level criteria; Pelvic fracture due to trauma, not otherwise meeting level criteria; Pregnancy (>20 weeks) with abdominal trauma; Significant injury with existing active medical co-morbidity or extremes of age; Admission for care of acute injury related to known or suspected child physical abuse (non-accidental trauma/NAT); Discretion of ED attending; Single system injury with significant mechanism. Institutional triage criteria encompass mechanisms associated with significant traumatic injury, including, 1) History of partial or complete ejection or rollover; 2) Fall greater than 10 feet (all ages); 3) Death in same passenger compartment; 4) Pedestrian/bicycle rider thrown, run over or with significant impact; 5) History of high speed crash with significant vehicular intrusion; 6) Need for extrication for entrapped patient; 7) Rider separated from transport vehicle with significant impact (motorcycle, ATV, Horse, etc); 8) Explosion or blast injury.

### II. METHOD OF TRAINING A MACHINE LEARNING MODEL TO PREDICT THE ASSIGNMENT OF MEDICAL RESOURCES REQUIRED TO TREAT A PATIENT FOR TRAUMATIC INJURY

One aspect of the present application relates to a method of training a machine learning model to predict the assignment of medical resources required to treat a patient for traumatic injury, the method comprising: (a) establishing a ground-truth, wherein the ground-truth corresponds to a standardized trauma activation level for a standardized patient with a standardized set of clinical conditions; (b) assigning a first trauma activation level for an individual patient identified with a plurality of clinical conditions selected from the set of clinical conditions, wherein a machine learning model assigns the first trauma activation level with an algorithm containing a plurality of relative calculable values, wherein each relative calculable value is assigned to one or more clinical conditions in the standardized set of clinical conditions; (c) determining, by human medical review, for the individual patient which of the set of clinical conditions with which the individual patient presented; (d) determining, by human medical review, a second trauma activation level that matches the set of clinical conditions that the individual patient presented; (e) inputting into the machine learning model the second trauma activation level, determined by human medical review, based on the set of clinical conditions that the individual patient presented; (f) comparing the second trauma activation level, determined by human medical review, based on the set of clinical conditions that the individual patient presented to the first trauma activation level, assigned by the machine learning model, for an individual patient identified with a plurality of clinical conditions selected from the set of clinical conditions; (g) determining, based on said comparison, whether the first trauma activation level assigned by the machine learning model corresponds to either under-triage, over-triage, or an appropriate level of triage for the individual patient in a clinical environment; (h) comparing the second trauma activation level, determined by human medical review, based on the set of clinical conditions that the individual patient presented to the ground-truth; (i) determining, based on said comparison, whether the second trauma activation level corresponds to either under-triage, over-triage, or an appropriate level of triage for the individual patient in a clinical environment; (j) adjusting one or more relative calculable values used by the machine learning model in assigning the first trauma activation level in response to the determination of whether the individual patient received either under-triage, over-triage, or an appropriate level of triage for the individual patient in a clinical environment; and (k) repeating steps (b) to (j) with a plurality of training patients, each identified with a plurality of clinical conditions selected from the set of clinical conditions until the machine learning model reaches desired parameters in step (I); and (I) assigns a third trauma activation level for a plurality of testing patients, each identified with a plurality of clinical conditions selected from the standardized set of clinical conditions which corresponds to an appropriate level of triage within desired parameters.

In particular embodiments, the desired parameters include a machine accuracy rate, wherein the machine accuracy rate is determined by: (1) assigning a third trauma activation level, by the machine learning model, for a plurality of testing patients, each identified with a plurality of clinical conditions selected from the standardized set of clinical conditions; (2) comparing the third trauma activation level for each testing patient to the ground-truth and determining, based on said comparison, whether the third trauma activation level assigned by the machine learning model corresponds to under-triage, over-triage, or an appropriate level of triage for each testing patient; and (3) calculating the machine accuracy rate for the testing patients based on the following formula

$$Machine Accuracy Rate = \frac{Number of testing patients with appropriate level of triage}{Number of total testing patients} \times 100 \%$$

In certain embodiments, the machine accuracy rate equals to, or greater than, 70%, 75%, 80%, 85%, 90%, 95%, or 98%.

In some embodiments, the desired parameters include an under-triage rate and/or an over-triage rate. The under-triage rate and over-triage rate are determined by the following formulas:

$$Under - Triage Rate = \frac{Number of testing patients with under triage}{Number of total testing patients} \times 100 \%$$

$$Over - Triage Rate = \frac{Number of testing patients with over triage}{Number of total testing patients} \times 100 \%$$

wherein a trauma activation level assigned to a patient that is lower than a trauma activation level assigned to the same patient based the ground truth is considered under-triage and wherein a trauma activation level assigned to a patient that is higher than the trauma activation level assigned to the same patient based on the ground truth is considered over-triage.

In certain embodiments, the under-triage rate equals to, or lower than, 1%, 2%, 5%, 10%, 15%, 20%, 25% or 30%.

Emergency rooms make decisions about assigning medical resources on necessarily limited information that is acquired prior to the time of patient arrival from the ambulance team on the way to the hospital or information that is obtained upon arrival in the emergency room itself. The classification of patients is essential, with a risk that the wrong classifications can lead to under-triage, which is bad for the patient, or can lead to over-triage, which results in the misuse of limited hospital resources. As discussed herein, the determination of a ground-truth for these classifications is challenging, but necessary to establish a supervised learning model that has a reliable baseline for correct triage, so as to evaluate under-triage and over-triage instances. This demands clinical knowledge. The assignment of trauma team activation (TTA) level to match a specific patient with traumatic injuries can be conceptualized as a simple classification task in which a set of predetermined criteria are used in conjunction with available patient data to assign specific TTA level for each patient. Timely activation of the trauma system allows the injured patient to be met by the trauma team upon arrival, therefore activation of trauma system prior to patient arrival is encouraged, whenever possible.

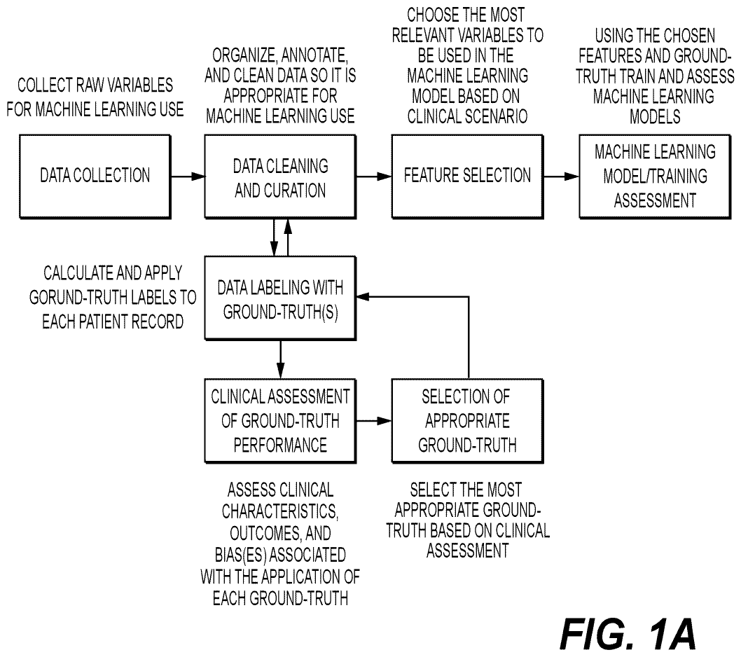

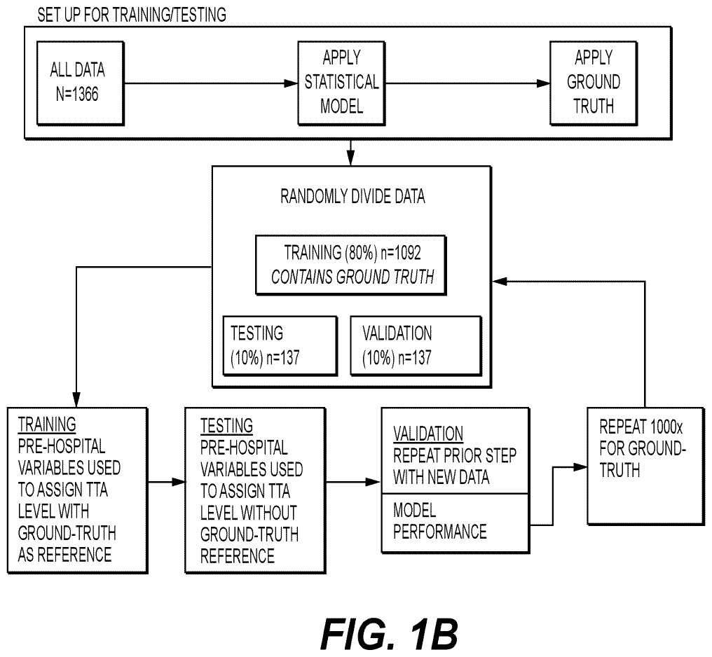

This application discloses how to determine the ideal ground-truth for training machine learning models to predict TTA level accurately in patients with traumatic injuries (FIG. 1, Panel A and B). In certain embodiments, combining the Cribari and Need for Trauma Intervention (NFTI) methods improves model performance compared to either method alone. Actual trauma team activation level was compared to recommended level classification by each ground-truth (Cribari, NFTI, or Cribari+NFTI). Demographics, pre-injury characteristics, and injury mechanisms were compared in cases of classification disagreement.

There are several parameters that can be used to determine model performance, including, but not limited to:
TP=true positive
FP=false positive
TN=true negative
FN=false negative
Precision=Positive predictive value: TP/(TP+FP)
Recall=Negative predictive value: TN/(TN+FN)
F1: harmonic mean of precision and recall=2*(Precision*Recall)/(Precision+Recall)
Accuracy=number of patients correctly classified (triaged)/total number of patients tested=(TP+TN)/(TP+FP+TN+FN)

This application identifies the ground-truth that can be used in predictive modeling; in particular, in a specific embodiment, hyper-parameter tuning (clinical selection of certain parameters as of particular significance) is used to bias the model away from under-triage (so as to avoid harm to the patient), even though this may result in instances of over-triage.

Machine learning, a subtype of artificial intelligence, can be used to optimize classification predictions based on features provided in a large dataset. Additional advantages of machine learning include the ability to handle complex data and to function with nonlinear and missing data. However, supervised machine learning approaches require that a correct answer or “ground-truth” be provided with the data used to train the model. The model learns patterns based on the data and the ground-truth and can make predictions or classifications based on those patterns.

The choice of ground-truth for TTA level is not straightforward as there is not a single gold-standard method for determining whether the TTA level was accurately assigned.

The Cribari matrix method is the most common and is based on the ACS COT optimal resources document definition of major trauma (patient with Injury Severity Score (ISS)>15). In clinical practice, the Cribari mode of classification only provides information retrospectively, and in effect adds up the different injuries that are identified after a patient reaches the hospital. The Cribari matrix assesses head or neck, face, chest, abdominal/pelvic contents, extremities/pelvic girdle, and external injuries, according to an injury severity score (1-75) calculated from the highest abbreviated injury scale (AIS) code in each of the three most severe regions (full TTA for a score of 16-75).

In various embodiments, alternative scoring systems may be used, including the Need for Trauma Intervention (NFTI), which is more strongly associated with outcomes after trauma than the ISS. The NFTI approach uses six criteria, when more than one is present, full trauma team activation occurs. NFTI assesses: 1) Receiving packed red blood cells within 4 hrs of arrival to the emergency department (ED), 2) Discharge from the emergency department (ED) to the operating room within 90 minutes of arrival, 3) Discharge from the ED to interventional radiology, 4) Discharge from the ED to the intensive care unit (ICU) with an ICU length of stay (LOS) of 3 or more days, (5) Mechanical ventilation outside of procedural anesthesia within 3 days of arrival, and (6) Death within 60 hours of arrival.

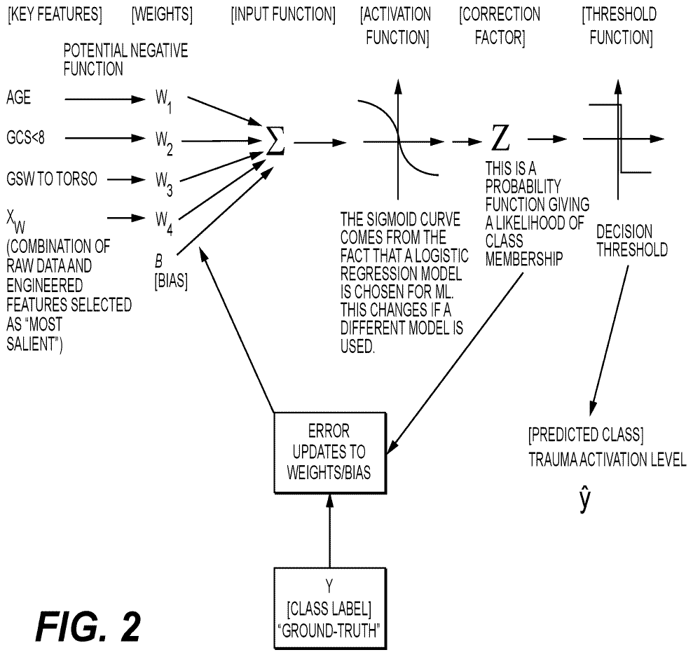

Clinically, a combination of Cribari and NFTI performs best, but, as discussed herein, this does not necessarily hold true when machine learning is used. Therefore, herein weighted parameters are used in the model to enhance the machine learning model based on clinical knowledge. FIG. 2 shows how the feature weights relate to the output from the algorithm.

Another aspect of the present application relates to a method of training a machine learning model to predict the assignment of medical resources required to treat a patient for traumatic injury, the method comprising the steps of (a) establishing a ground-truth, wherein the ground-truth corresponds to a standardized trauma activation level for a standardized patient with a standardized set of clinical conditions; (b) assigning a machine trauma activation level for an individual patient identified with a plurality of clinical conditions selected from the standardized set of clinical conditions, wherein a machine learning model assigns the first trauma activation level with an algorithm containing a plurality of relative calculable values, wherein each relative calculable value is assigned to one or more clinical conditions in the standardized set of clinical conditions; (c) comparing the machine trauma activation level to the ground-truth; (d) determining, based on said comparison, whether the machine trauma activation level corresponds to under-triage, over-triage, or an appropriate level of triage for the individual patient; (e) adjusting one or more relative calculable values used by the machine learning model in assigning the machine trauma activation level if the machine trauma activation level corresponds to under-triage or over-triage in step (d); and (f) repeating steps (b) to (e) with a plurality of patients, each identified with a plurality of clinical conditions selected from the set of clinical conditions until the machine learning model reaches an appropriate level of triage within desired parameters. In some embodiments, the ground-truth is based on the ISS, the NFTI, or a combination of the ISS and the NFTI. In some embodiments, the individual patient has a previous trauma activation level assigned by a human medical service provider. In some embodiments, the desired parameters include a machine accuracy rate.

The methods described herein can be used for both adult and pediatric patients. However, models developed for pediatric treatment must be developed based on pediatric populations; child patients cannot be considered equivalent to adult patients. Different hyper-parameter tuning can be used for pediatric populations.

It is a particular feature of the methods of this application that they rely on raw data obtained pre-hospital or in hospital emergency departments, and that this raw data is then used for feature selection when classifying patients for an appropriate level of triage.

It is another particular feature of the methods of this application that hyper-parameter tuning of the various features that are selected for purposes of classifying patients for triage is used to shift the logic within the model used (e.g., logistic regression model).

The model can be designed for self-improvement, e.g., by deep learning. The use of deep learning introduces a level of internalized quality assurance check for the algorithm assigning medical resources.

An artificial intelligence module may comprise or function as artificial intelligence logic stored in memory which may be executable by the processor, of one or more servers and/or client devices. In some embodiments, the artificial intelligence module may function as or comprise a machine/deep learning/artificial intelligence platform that interrogates the healthcare information or data of the system and learns about healthcare behaviors and trends of one or more patients.

In further embodiments, the artificial intelligence module may function to provide and recommend solutions, such as therapies which are cost effective and which may successfully treat a condition of a patient, to patients and healthcare providers.

In still further embodiments, the artificial intelligence module may function to generate population data and other informatics, such as anonymized general patient population data, for healthcare organizations and Pharma using information of one or more patients stored in one or more data stores, and/or blockchain databases.

### III. METHOD FOR ASSIGNING MEDICAL RESOURCES TO A SUBJECT FOR TREATMENT OF TRAUMATIC INJURY

Another aspect of the application relates to a method for assigning medical resources to a subject for treatment of traumatic injury. The method comprises the steps of (a) receiving, via a user interface of an application executing on one or more computer processors, a personal information profile, wherein said personal information profile comprises inputs for personal information for a plurality of types of information selected from the types of information comprising comorbidities, engineered features, prehospital interventions, injury mechanisms and numeric variables; (b) storing, via the one or more computer processors, said personal information of said subject in a database accessible by said application, and accessible by said subject via said user interface of said application; (c) receiving, via said user interface of said application executing on one or more computer processors, a data visualization dashboard, wherein said dashboard displays predictions of medical resource requirements in response to said personal information stored in said database; (d) assigning, via the one or more computer processors, a calculable value for any of a plurality of the types of information in said personal information stored in said database and a calculable value point matrix stored on a memory device accessible by the one or more computer processors, the calculable value point matrix being generated and continually updated by a machine learning module; (e) determining, via the one or more computer processors, a total calculable value of said types of information in said personal information for said subject via the one or more computer processors, based on evaluation in real-time, wherein when the total calculable value of said types of information in said personal information for said subject exceeds or falls below a pre-selected calculable value a predicted assignment of medical resources is generated; (f) receiving, via a user interface of an application executing on one or more computer processors, a goal-setting module, wherein said goal-setting module comprises inputs from a medical professional regarding calculable values to be assigned to types of information in said personal information that are focuses for clinical care based on the personal information stored in said database; (g) notifying, via said user interface, a medical professional providing medical care to said subject about the predicted assignment of medical resources for said subject.

Another aspect of the application relates to a method for assigning medical resources to a subject for treatment of traumatic injury. The method comprises the steps of (a) receiving, via a user interface of an application executing on one or more computer processors, a personal information profile, wherein said personal information profile comprises inputs for personal information for a plurality of types of information selected from the types of information comprising comorbidities, engineered features, prehospital interventions, injury mechanisms and numeric variables; (b) storing, via the one or more computer processors, said personal information of said subject in a database accessible by said application, and accessible by said subject via said user interface of said application; (c) assigning, via the one or more computer processors, a calculable value for any of a plurality of the types of information in said personal information stored in said database and a calculable value point matrix stored on a memory device accessible by the one or more computer processors, the calculable value point matrix being generated and continually updated by a machine learning module; (d) determining, via the one or more computer processors, a total calculable value of said types of information in said personal information for said subject via the one or more computer processors, based on evaluation in real-time, wherein when the total calculable value of said types of information in said personal information for said subject exceeds or falls below a pre-selected calculable value a predicted assignment of medical resources is generated; and (e) notifying, via said user interface, a medical professional providing medical care to said subject about the predicted assignment of medical resources for said subject.

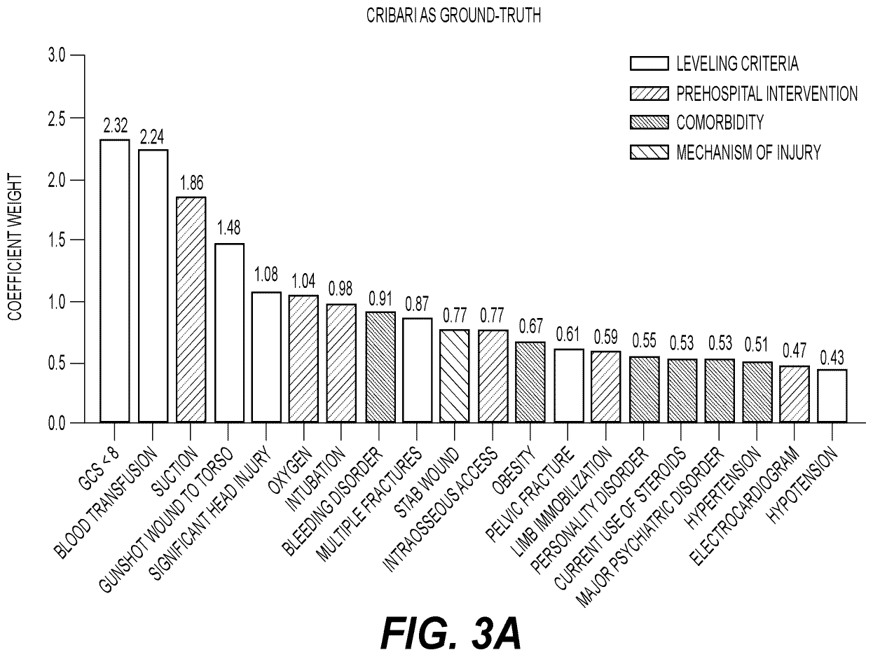

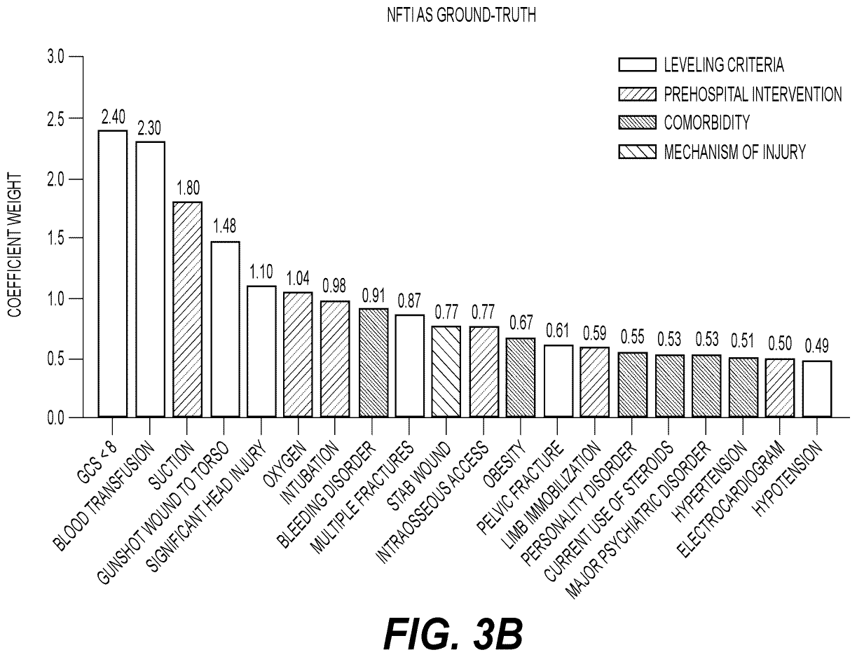

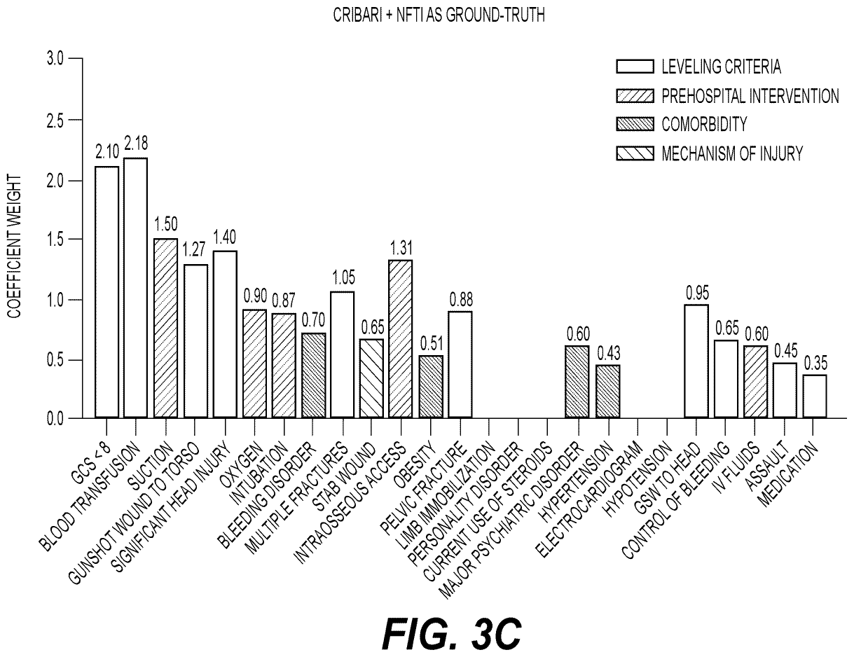

Assignment of medical resources is based on raw data regarding features of the patient identified by pre-hospital (e.g., ambulance team or other first responders) or hospital staff (e.g., emergency room nurse). The models for assigning resources herein do not necessarily sum weight values given to features (although in certain circumstances this may be done); however, the models do take into account the relative values of assigned weights. An evaluation of weight values occurs in real-time in the model; this creates a weighted feature map, but this is not necessarily used as part of the algorithm assigning medical resources. Features that are assessed include, but are not limited to, comorbidities, mechanisms of injury, and pre-hospital interventions (see, e.g., Table 4 and FIG. 3, Panel A, B and C).

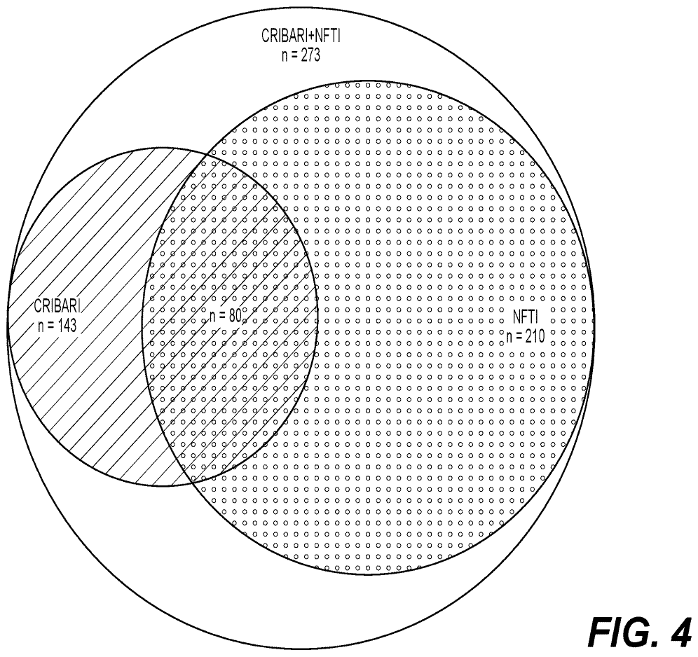

The raw data regarding patients can be obtained pre-hospital, in-hospital, or both; however, pre-work variable manipulation requires specialist clinical knowledge. The minimization of under-triage in the application of the model to assign medical resources in real time requires post-work hyperparameter tuning to bias the model's outcomes away from under-triage. In certain embodiments, different algorithms may be used to build a supervised learning classifier (FIG. 4). In certain embodiments, different classifiers may be used in the methods and systems herein (e.g., logistic regression, decision tree, random forest, support vector machine, K Nearest Neighbor, Naïve Bayes). In preferred embodiments, logistic regression and random forest are used.

In a certain embodiment, a method of assigning medical resources to a subject who is a patient brought to an emergency room uses a series of standard questions for emergency responders and/or hospital staff to answer concerning the subject (e.g., ten questions). Based on these questions, the model developed via the training methods described herein assigns medical resources with a designed bias away from the risk of under-triage. The model is trained so that if four or more the questions can be answered then as assignment of medical resources can occur within high confidence intervals for an appropriate level of triage.

A graphical user interface (GUI) is provided to users (e.g., emergency room staff, etc). In certain embodiments, the graphical user interface will have less than twenty variables for users to enter in information to obtain a predictive assessment of medical resources required. The graphical user interface can operate via dropdown boxes and other standard options known to be provided by GUIs.

### IV. SYSTEM FOR ASSIGNING MEDICAL RESOURCES TO A SUBJECT FOR TREATMENT OF TRAUMATIC INJURY IN A SUBJECT

Another aspect of the application relates to a system for assigning medical resources to a subject for treatment of traumatic injury in a subject. The system comprises one or more computer processors; and one or more tangible computer readable media accessible by the one or more computer processors, wherein the one or more tangible computer readable media comprise instructions that, when executed by the one or more processors, cause the one or more processors to perform: (a) receiving, via a user interface of an application executing on one or more computer processors, a personal information profile, wherein said personal information profile comprises inputs for personal information for a plurality of types of information selected from the types of information comprising comorbidities, engineered features, prehospital interventions, injury mechanisms and numeric variables; (b) storing, via the one or more computer processors, said personal information of said subject in a database accessible by said application, and accessible by said subject via said user interface of said application; (c) receiving, via said user interface of said application executing on one or more computer processors, a data visualization dashboard, wherein said dashboard displays predictions of medical resource requirements in response to said personal information stored in said database; (d) assigning, via the one or more computer processors, a calculable value for any of a plurality of the types of information in said personal information stored in said database and a calculable value point matrix stored on a memory device accessible by the one or more computer processors, the calculable value point matrix being generated and continually updated by a machine learning module; (e) determining, via the one or more computer processors, a total calculable value of said types of information in said personal information for said subject via the one or more computer processors, based on evaluation in real-time, wherein when the total calculable value of said types of information in said personal information for said subject exceeds or falls below a pre-selected calculable value a predicted assignment of medical resources is generated; (f) receiving, via a user interface of an application executing on one or more computer processors, a goal-setting module, wherein said goal-setting module comprises inputs from a medical professional regarding calculable values to be assigned to types of information in said personal information that are focuses for clinical care based on the personal information stored in said database; (g) notifying, via said user interface, a medical professional providing medical care to said subject about the predicted assignment of medical resources for said subject.

Another aspect of the application relates to a system for assigning medical resources to a subject for treatment of traumatic injury in a subject. The system comprises one or more computer processors; and one or more tangible computer readable media accessible by the one or more computer processors, wherein the one or more tangible computer readable media comprise instructions that, when executed by the one or more processors, cause the one or more processors to perform: (a) receiving, via a user interface of an application executing on one or more computer processors, a personal information profile, wherein said personal information profile comprises inputs for personal information for a plurality of types of information selected from the types of information comprising comorbidities, engineered features, prehospital interventions, injury mechanisms and numeric variables; (b) storing, via the one or more computer processors, said personal information of said subject in a database accessible by said application, and accessible by said subject via said user interface of said application; (c) assigning, via the one or more computer processors, a calculable value for any of a plurality of the types of information in said personal information stored in said database and a calculable value point matrix stored on a memory device accessible by the one or more computer processors, the calculable value point matrix being generated and continually updated by a machine learning module; (d) determining, via the one or more computer processors, a total calculable value of said types of information in said personal information for said subject via the one or more computer processors, based on evaluation in real-time, wherein when the total calculable value of said types of information in said personal information for said subject exceeds or falls below a pre-selected calculable value a predicted assignment of medical resources is generated; and (e) notifying, via said user interface, a medical professional providing medical care to said subject about the predicted assignment of medical resources for said subject.

It will be appreciated that some exemplary embodiments described herein may include one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the methods and/or systems described herein. Alternatively, some or all functions may be implemented by a state machine that has no stored program instructions, or in one or more application specific integrated circuits (ASICs), in which each function or some combinations of certain of the functions are implemented as custom logic. Of course, a combination of the two approaches may be used. Moreover, some exemplary embodiments may be implemented as a computer-readable storage medium having computer readable code stored thereon for programming a computer, server, appliance, device, etc. each of which may include a processor to perform methods as described and claimed herein. Examples of such computer-readable storage mediums include, but are not limited to, a hard disk, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory), a Flash memory, and the like.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible program carrier for execution by, or to control the operation of, data processing apparatus. The tangible program carrier can be a propagated signal or a computer readable medium. The propagated signal is an artificially generated signal, e.g., a machine generated electrical, optical, or electromagnetic signal that is generated to encode information for transmission to suitable receiver apparatus for execution by a computer. The computer readable medium can be a machine readable storage device, a machine readable storage substrate, a memory device, a composition of matter effecting a machine readable propagated signal, or a combination of one or more of them.

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, solid state drives, or optical disks. However, a computer need not have such devices.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network or the cloud. The relationship of client and server arises by virtue of computer programs running on the respective computers and having a client server relationship to each other.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

The computer system may also include a main memory, such as a random access memory (RAM) or other dynamic storage device (e.g., dynamic RAM (DRAM), static RAM (SRAM), and synchronous DRAM (SDRAM)), coupled to the bus for storing information and instructions to be executed by processor. In addition, the main memory may be used for storing temporary variables or other intermediate information during the execution of instructions by the processor. The computer system may further include a read only memory (ROM) or other static storage device (e.g., programmable ROM (PROM), erasable PROM (EPROM), and electrically erasable PROM (EEPROM)) coupled to the bus for storing static information and instructions for the processor.

The computer system may also include a disk controller coupled to the bus to control one or more storage devices for storing information and instructions, such as a magnetic hard disk, and a removable media drive (e.g., floppy disk drive, read-only compact disc drive, read/write compact disc drive, compact disc jukebox, tape drive, and removable magneto-optical drive). The storage devices may be added to the computer system using an appropriate device interface (e.g., small computer system interface (SCSI), integrated device electronics (IDE), enhanced-IDE (E-IDE), direct memory access (DM A), or ultra-DMA).

The computer system may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGA s)).

The computer system may also include a display controller coupled to the bus to control a display, such as a cathode ray tube (CRT), liquid crystal display (LCD) or any other type of display, for displaying information to a computer user. The computer system may also include input devices, such as a keyboard and a pointing device, for interacting with a computer user and providing information to the processor. Additionally, a touch screen could be employed in conjunction with display. The pointing device, for example, may be a mouse, a trackball, or a pointing stick for communicating direction information and command selections to the processor and for controlling cursor movement on the display. In addition, a printer may provide printed listings of data stored and/or generated by the computer system.

The computer system performs a portion or all of the processing steps of the invention in response to the processor executing one or more sequences of one or more instructions contained in a memory, such as the main memory. Such instructions may be read into the main memory from another computer readable medium, such as a hard disk or a removable media drive. One or more processors in a multi-processing arrangement may also be employed to execute the sequences of instructions contained in main memory. In alternative embodiments, hard-wired circuitry may be used in place of or in combination with software instructions. Thus, embodiments are not limited to any specific combination of hardware circuitry and software.

As stated above, the computer system includes at least one computer readable medium or memory for holding instructions programmed according to the teachings of the invention and for containing data structures, tables, records, or other data described herein. Examples of computer readable media are compact discs, hard disks, floppy disks, tape, magneto-optical disks, PROM s (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave (described below), or any other medium from which a computer can read.

### V. TANGIBLE NON-TRANSITORY COMPUTER READABLE STORAGE MEDIUM

Another aspect of the application relates to a tangible non-transitory computer readable storage medium that comprises instructions that, when executed by a computer processor, cause the processor to: (a) receiving, via a user interface of an application executing on one or more computer processors, a personal information profile, wherein said personal information profile comprises inputs for personal information for a plurality of types of information selected from the types of information comprising comorbidities, engineered features, prehospital interventions, injury mechanisms and numeric variables; (b) storing, via the one or more computer processors, said personal information of said subject in a database accessible by said application, and accessible by said subject via said user interface of said application; (c) receiving, via said user interface of said application executing on one or more computer processors, a data visualization dashboard, wherein said dashboard displays predictions of medical resource requirements in response to said personal information stored in said database; (d) assigning, via the one or more computer processors, a calculable value for any of a plurality of the types of information in said personal information stored in said database and a calculable value point matrix stored on a memory device accessible by the one or more computer processors, the calculable value point matrix being generated and continually updated by a machine learning module; (e) determining, via the one or more computer processors, a total calculable value of said types of information in said personal information for said subject via the one or more computer processors, based on evaluation in real-time, wherein when the total calculable value of said types of information in said personal information for said subject exceeds or falls below a pre-selected calculable value a predicted assignment of medical resources is generated; (f) receiving, via a user interface of an application executing on one or more computer processors, a goal-setting module, wherein said goal-setting module comprises inputs from a medical professional regarding calculable values to be assigned to types of information in said personal information that are focuses for clinical care based on the personal information stored in said database; (g) notifying, via said user interface, a medical professional providing medical care to said subject about the predicted assignment of medical resources for said subject.

In some embodiments, the tangible non-transitory computer readable storage medium described herein, further comprises instructions that, when executed by a computer processor, cause the processor to: (i) enable one or more computing devices in data communication with each other, each device having one or more computer processors, a data communication connection, and one or more tangible non-transitory computer-readable media accessible by the one or more computer processors, (ii) store a personal information database; and (iii) inputting into a machine learning module, wherein the personal information database and the machine learning module are each stored in the one or more tangible non-transitory computer-readable media.

A computer program (also known as a program, software, software application, application, script, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Additionally, the particular methods and/or corresponding acts in support of steps and corresponding functions described herein, may also be utilized to implement corresponding software structures and algorithms, and equivalents thereof. The processes described in this specification can be performed by one or more programmable processors (computing device processors) executing one or more computer applications or programs to perform functions by operating on input data and generating output.

Stored on any one or on a combination of computer readable media, the present invention includes software for controlling the computer system, for driving a device or devices for implementing the invention, and for enabling the computer system to interact with a human user. Such software may include, but is not limited to, device drivers, operating systems, development tools, and applications software. Such computer readable media further includes the computer program product of the present invention for performing all or a portion (if processing is distributed) of the processing performed in implementing the invention.

The computer code or software code of the present invention may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

Various forms of computer readable media may be involved in carrying out one or more sequences of one or more instructions to processor for execution. For example, the instructions may initially be carried on a magnetic disk of a remote computer. The remote computer can load the instructions for implementing all or a portion of the present invention remotely into a dynamic memory and send the instructions over the air (e.g., through a wireless cellular network or WiFi network). A modem local to the computer system may receive the data over the air and use an infrared transmitter to convert the data to an infrared signal. An infrared detector coupled to the bus can receive the data carried in the infrared signal and place the data on the bus. The bus carries the data to the main memory, from which the processor retrieves and executes the instructions. The instructions received by the main memory may optionally be stored on storage device either before or after execution by processor.

The computer system also includes a communication interface coupled to the bus. The communication interface provides a two-way data communication coupling to a network link that is connected to, for example, a local area network (LAN), or to another communications network such as the Internet. For example, the communication interface may be a network interface card to attach to any packet switched LAN. As another example, the communication interface may be an asymmetrical digital subscriber line (ADSL) card, an integrated services digital network (ISDN) card or a modem to provide a data communication connection to a corresponding type of communications line. Wireless links may also be implemented. In any such implementation, the communication interface sends and receives electrical, electromagnetic or optical signals that carry digital data streams representing various types of information.

The network link typically provides data communication to the cloud through one or more networks to other data devices. For example, the network link may provide a connection to another computer or remotely located presentation device through a local network (e.g., a LAN) or through equipment operated by a service provider, which provides communication services through a communications network. In preferred embodiments, the local network and the communications network preferably use electrical, electromagnetic, or optical signals that carry digital data streams. The signals through the various networks and the signals on the network link and through the communication interface, which carry the digital data to and from the computer system, are exemplary forms of carrier waves transporting the information. The computer system can transmit and receive data, including program code, through the network(s) and, the network link and the communication interface. Moreover, the network link may provide a connection through a LAN to a client device or client device such as a personal digital assistant (PDA), laptop computer, tablet computer, smartphone, or cellular telephone. The LAN communications network and the other communications networks such as cellular wireless and wifi networks may use electrical, electromagnetic or optical signals that carry digital data streams. The processor system can transmit notifications and receive data, including program code, through the network(s), the network link and the communication interface.

The present application is further illustrated by the following examples that should not be construed as limiting. The contents of all references, patents, and published patent applications cited throughout this application, as well as the Figures and Tables, are incorporated herein by reference.

### EXAMPLES

### Example 1: Machine Learning Predictions of Pediatric Trauma Activation Level Vary According to Ground-Truth

METHODS: Retrospective data were collected from the institutional trauma registry for all pediatric patients who triggered a trauma team activation between January 2014 and January 2020 at the Pediatric Trauma Center in Western New York. No patients were excluded from the analysis.

Data collected included: patient demographics, mechanism of injury, medical comorbidities, prehospital interventions, prehospital Glasgow Coma Score (GCS), prehospital revised trauma score (RTS), prehospital systolic blood pressure (SBP), initial GCS in the ED, and initial SBP in the ED. Additional features were derived from the original dataset based on current institutional trauma team activation criteria. In addition, injury severity score (ISS) and 6 NFTI criteria8 were collected, including: 1) transfusion of packed red blood cells within the first 4 hours of arrival; 2) transfer from the Emergency Department (ED) to the operating room within 90 min; 3) transfer from the ED to Interventional Radiology (IR); 4) transfer from the ED to the Intensive Care Unit (ICU) with an ICU length of stay ≥3 days; 5) mechanical ventilation excluding procedural anesthesia within 3 days; and 6) death within 60 hours of arrival. Trauma Team Activation (TTA) was modeled with two levels: full and partial activation.

Three ground-truths were constructed for prediction modeling: 1) Cribari method: ISS >15 required full activation; 2) NFTI: positive for any of the 6 NFTI criteria required full activation; 3) Cribari+NFTI: ISS >15 or positive for any of the 6 NFTI criteria required full activation. To determine over- and undertriage, a TTA level was assigned to each patient based on each ground-truth and the ground-truth TTA level was compared to the actual activation level assigned by ED staff.

For machine learning, the dataset was randomly divided into training (80%), validation (10%), and testing (10%) data. Two explainable classical machine learning models (Logistic Regression and Random Forest) were trained/tested 1000 times in separate trials with each of the three ground-truths. The hyperparameters of each model were tuned using model performance on the validation set. Continuous data are presented as mean (standard deviation [SD]) or median (interquartile range), as appropriate. Categorical variables are presented as percentage (number [n]). Model performance was assessed using the area under the receiver operating characteristic curve (AUC) and 95% confidence intervals (CI) were estimated over the 1000 trials.

RESULTS: Of 1366 patients included, 230 (16.8%) triggered a full activation. Table 1 shows the proportions of patients identified as under-, over-, and correctly triaged for each ground-truth.

TABLE 1
Triage Classification with Three Ground-Truths

Triage

| Classifi- | | | | | p- | Cribari + | |
|---|---|---|---|---|---|---|---|
| cation | Cribari | | NFTI | | value* | NFTI | |
| Correct | 80.2% | (1096) | 79.1% | (1080) | 0.476 | 75% | (1024) |
| Under | 10.5% | (143) | 15.4% | (210) | <0.001 | 20% | (273) |
| Over | 9.3% | (127) | 5.6% | (76) | <0.001 | 5.1% | (69) |

*chi-square test comparing Cribari to NFTI.

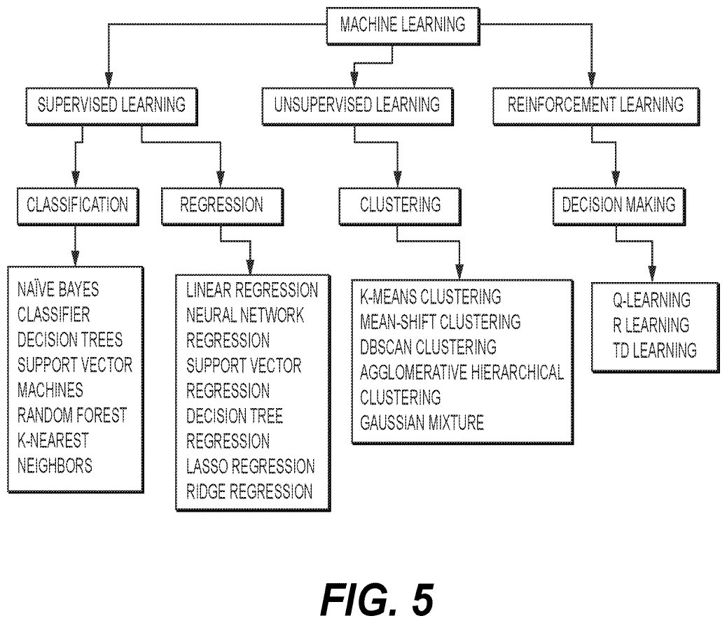

Cribari and NFTI identified different, but overlapping sets of patients that were under-triaged. For example, of the 210 patients identified as under-triaged by NFTI, only 38.1% (80) were also identified as under-triaged by Cribari (FIG. 5). Therefore, Cribari+NFTI captured significantly more patients that were under-triaged, compared to either method alone. Similarly, different but overlapping sets of patients were also identified as over-triaged. Patients identified as under-triaged were compared between the three groups (Table 2). NFTI detected significantly more under-triaged children with penetrating mechanisms of injury compared to Cribari. In addition, the combination of Cribari+NFTI captured more patients with a penetrating mechanism of injury, compared to either method alone (Table 2).

TABLE 2
Patient Characteristics in Under-Triage with Three Ground-Truths

| | Cribari | | NFTI Under- | | | Cribari + NFTI | |
|---|---|---|---|---|---|---|---|
| | Under-triage | | triage | | | Under-triage | |
| | (n = 143) | | (n = 210) | | p-value* | (n = 273) | |
| Penetrating | 80.2% | (1096) | 79.1% | (1080) | 0.476 | 75% | (1024) |
| Mechanism: % (n) | | | | | | | |
| Stab Wounds: % (n) | 10.5% | (143) | 15.4% | (210) | <0.001 | 20% | (273) |
| Mortality: % (n) | 9.3% | (127) | 5.6% | (76) | <0.001 | 5.1% | (69) |

*chi-square test comparing Cribari to NFTI.

When specific mechanisms of injury were evaluated, NFTI detected more under-triaged children with stab wounds compared to Cribari. There were no other significant differences for individual mechanisms of injury. Patients with stab wounds that were under-triaged per NFTI had a mean ISS of 6.2 (8.6), demonstrating why more were missed by Cribari. Furthermore, 75% of these patients went to the operating room within 90 minutes of arrival. All three ground-truths captured a similar proportion of mortality in under-triaged patients (Table 2). However, of the 46 patients who died (mortality rate 3.4% overall), Cribari only captured 67.4% (31), while NFTI and Cribari+NFTI captured 97.8% (45). All machine learning models outperformed TTA determination by ED staff. ED staff classification showed modest accuracy with higher variability compared to machine learning models and was most accurate with NFTI as ground-truth (Table 3).

TABLE 3
Machine Learning Model Performance with Three Ground-Truths

| Prediction | Cribari | NFTI | Cribari + NFTI |
|---|---|---|---|
| ED Staff | 65.27 ± 0.04 | 65.36 ± 0.04 | 64.85 ± 0.03 |
| Logistic | 85.52 ± 1.1e⁻¹⁶ | 80.02 ± 1.1e⁻¹⁶ | 78.46 ± 0.00 |
| Regression | | | |
| Random | 84.81 ± 2.0e⁻³ | 79.64 ± 0.00 | 80.44 ± 1.3e⁻³ |
| Forest | | | |

With respect to pure model performance, logistic regression using Cribari as ground-truth performed best. However, as mentioned above, each ground-truth identified a different subset of patients who met criteria for a full activation.

Trauma systems have reduced mortality after severe trauma and are dependent upon efficient systems that accurately match the needs of the injured patient with appropriate hospital resources and services. The results of this study highlight the difficulty associated with optimizing TTA criteria, even within a single institution. This work demonstrates that the two most common methods of retrospective triage assessment differ in their ability to detect specific injury characteristics. Specifically, NFTI is more sensitive for penetrating mechanisms of injury, where the cumulative injury burden is not enough to raise the ISS above 15.

However, the injury burden is significant enough that 75% of the patients with stab wounds designated as under-triaged by NFTI required operative intervention within 90 minutes of arrival.

Furthermore, NFTI caught 97.8% of the mortality associated with under-triage in this pediatric population, while Cribari only caught 67.4%. These results show that use of Cribari+NFTI for ground-truth is better than either method alone since under-triage would likely have worse adverse consequences than over-triage.

Reflecting the complexity of the triage process, all machine learning models in this study outperformed ED staff, regardless of the ground-truth used.

This shows that machine learning tools are helpful in the setting of hospital triage.

The Cribari+NFTI ground-truth degraded machine learning model performance, compared to the two methods alone. However, this combination captured more clinically important information, making this the more ideal ground-truth. This is an unexpected result.

The logistic regression model performed best when Cribari was used for ground-truth. This may be due logistic regression being better able to extrapolate a continuous measure of injury severity, compared to 6 binary responses with NFTI. Logistic regression models work well with binary data (such as used in Cribari's ISS scores); however, pure model performance is not equivalent to clinical relevance.

In conclusion, ideal TTA guidelines are succinct and capture all patients who truly require the highest level of trauma activation and none of those who do not.

These results show that machine learning can be used to help optimize TTA decisions in the emergency department, although clinical judgement and experience are critical even at the stages of model-building, to ensure performance metrics are grounded in the real-world.

### Example 2: Deep Learning Models for Prediction of Trauma Activation Level

Results demonstrate the feasibility of using machine-learning approaches for assignment of trauma activation level, thus, incorporation of deep learning will increase the robustness of the algorithm to missing data, a challenge often encountered in real world clinical scenarios. Results show that an interpretable algorithm performs within acceptable standards, but no comparisons have been made between deep learning models and existing triage accuracy or to the results from traditional interpretable ML models. Therefore, a retrospective study compares the performance of 3 deep learning models [1. convolutional neural network (CNN), 2. generative adversarial network (GAN), 3. Autoencoder] to the interpretable ML model developed herein. Model performance is assessed using accuracy, recall, precision, area under the receiver operating characteristic curve (AUC), and 95% confidence intervals (CI). Based on the data, a successful system achieves AUC≥80, 95% CI <±0.04, while minimizing under-triage.

Retrospective data is collected from an institutional trauma registry for all pediatric patients who triggered a trauma activation (1/2014-1/2022). Data collected includes: patient demographics, mechanism of injury, ICD9/10 codes, medical comorbidities, prehospital interventions, prehospital Glasgow Coma Score (GCS), prehospital revised trauma score (RTS), prehospital systolic blood pressure (SBP), initial GCS in the ED, initial SBP in the ED, ISS, and the 6 NFTI criteria (1. transfusion of packed red blood cells within the first 4 hours of arrival; 2. transfer from the ED to the operating room within 90 min; 3. transfer from the ED to Interventional Radiology (IR); 4. transfer from the ED to the Intensive Care Unit (ICU) with an ICU length of stay ≥3 days; 5. mechanical ventilation excluding procedural anesthesia within 3 days; and 6. death within 60 hours of arrival). ML features (Table 4) is extracted from the variables collected and additional features are engineered based on institutional trauma activation criteria.

TABLE 4
Predictors available to the machine learning model

| ENGINEERED FEATURES | PREHOSPITAL INTERVENTIONS |
|---|---|
| A cute injury related to known or | Airway Management (any of the below) |
| suspected child abuse | |
| Blood transfusion | Bag-valve mask ventilation |
| GCS <8 | Intubation |
| GSW to neck, torso, head, arm, leg, | Airway suction |
| or groin and buttock | |
| Hypotension | Placement of a laryngeal mask airway |
| Multiple rib fractures or flail chest | Placement of a supraglottic assist device |
| Pelvic fracture | Placement of an oral airway |
| Significant head injury with torso or | Bleeding Control (any of the below) |
| extremity trauma | |
| Stab wound to neck, torso, head, or | Tourniquet |
| groin and buttock | |
| Traumatic amputation proximal to wrist | Manual pressure |
| or ankle | |
| Two or more suspected proximal bone | Placement of pelvic binder |
| fractures | |
| | Hemostatic dressing |
| COMORBIDITIES | Cardiopulmonary Resuscitation (CPR) |
| Asthma | Electrocardiogram |
| Bleeding Disorder(s) (BldDo) | Intraosseous access |
| Cirrhosis or ascites | Intravenous (IV) fluids (any amount) |
| Current anticoagulation (WARFARIN) | <500 cc of IV fluids |
| Current smoker | <2000 cc of IV fluids |
| Current use of steroids (ROIDS) | >2000 cc of IV fluids |
| Diabetes mellitus | IV fluids (unknown amount) |
| Drug use disorder, substance abuse | Limb Immobilization |
| disorder, drug abuse | |
| Functional dependency (prior to | Medications, any |
| hospitalization) (DEP) | |
| History of congenital anomaly (CONG) | Needle thoracostomy |
| History of prematurity (PREMS) | Oxygen |
| History of psychiatric disorder (any | Spine Immobilization |
| of the below) (Psych) | |
| Attention deficit disorder (ADD)/ | |
| ADHD | |
| Personality disorder (PERSDO) | MECHANISMS OF INJURY |
| Major psychiatric disorder (not | Abuse |
| otherwise specified) (MajPsych) | |
| History of transplant (TXP) | Assault |
| Hypertension | Fall |
| Inflammatory bowel disease (e.g., | Gunshot wound (GSW) |
| Crohn's disease or ulcerative colitis) | |
| Obesity | Motor vehicle collision (MVC) |
| Pre-existing spinal cord injury | Other/unknown |
| Pre-hospital cardiac arrest (PHCPR) | Pedestrian struck |
| Renal failure or current dialysis (RF) | Sports-related injury |
| Seizures | Stab wound |

Trauma activation level is modeled using two levels: full and partial activation. Three ground-truths are used for prediction modeling: 1) Cribari method: ISS >15 indicates full activation; 2) NFTI: positive for any of the 6 NFTI criteria indicates full activation; 3) Cribari+NFTI: ISS >15 or positive for any of the 6 NFTI criteria indicates full activation. To determine over- and under-triage, a trauma activation level is assigned to each patient based on each ground-truth and subsequently compared to the actual activation level assigned by ED staff.

Specifically, when applied to an algorithm, the outcomes from the three sets of criteria: Cribari, NFTI, and Cribari+NFTI are applied as labels to each patient included in the training data. These labels then tell the computer the “right” answer or ground-truth for each patient. The ground-truth is used to adjust decision thresholds such that the computer can optimize the threshold to get the most correct answers possible, but ground-truths are not actually used as criteria for making the trauma team activation decision. Essentially, a ground-truth is used to check the algorithm's work, which is similar to how they are used in clinical practice.

Furthermore, regarding the cases in which Cribari fails because ISS is incomplete or missing, since the labels from Cribari are being used to check the algorithm's work, it is critically important to include the patients for whom ISS is incomplete or missing. As the goal is to determine the best criteria for training the algorithm, if Cribari were chosen, the algorithm would need to be able to handle these edge cases (i.e., adjust for these circumstances). If outliers such as these were removed, then when faced with such a case in a prospective manner, the algorithm would be more likely to fail. If these outliers lead the model to develop incorrect predictions for triage level, then Cribari is not the ideal criteria to use for training. Based on the studies discussed herein, the combination of Cribari+NFTI together mitigates this concern to some extent and creates the best criteria for training an algorithm to handle missing, inaccurate, or outlying data such as this.

For machine learning, the dataset is randomly divided into training (80%), validation (10%), and testing (10%) sets for each trial. Each of the three deep learning models (CNN, GAN, autoencoder) is trained/tested 1000 times in separate trials with each of the three ground-truths. The hyperparameters of each model are tuned using the validation set, balancing pure algorithm performance against clinical performance metrics (e.g., minimizing under-triage). Algorithm performance are assessed using accuracy, recall, precision, AUC, and 95% CIs constructed over the 1000 trials. Based on preliminary data, an optimal system achieves AUC≥80, 95% CI <±0.04, while minimizing under-triage rates to <12%.

Finally, the performance of all three deep learning algorithms is compared to the same performance metrics generated for the existing interpretable M L algorithm and to the real-world performance of human clinicians. The best of the algorithms, balancing both clinical measures and pure algorithm performance, is chosen for further testing. Based on the data, a successful deep learning algorithm outperforms human clinicians with respect to accuracy and variability of trauma activation level assignment, while meeting the above model performance standards.

### Example 3: a Graphical User Interface for Implementation

While myriad predictors are available for initial model development and tuning, algorithm performance depends most heavily on a weighted subset of variables or features. For implementation purposes, it is not practical to expect clinicians to input a large number of variables into the model prior to obtaining a predicted trauma activation level. A weighted feature map is generated for each of the models tested herein. Using the top 20 features for each model as predictors, retrospective predictions of trauma activation level assignment are performed and model performance is assessed. The optimal subset of features for each model are selected and fine-tuned to optimize prediction performance, balancing clinical concerns. Some degradation in model performance with a limited feature subset is expected. Therefore, a successful system continues to outperform human clinicians and will achieve AUC>85, 95% CI <0.04, while minimizing under-triage rates to <12%.

### Example 4: The Acceptability of Interpretable ML Vs. Deep Learning Models

The ultimate successful implementation of an algorithm depends on both model performance and the acceptability of that model to the target end-user. An investigation of algorithm acceptability is performed using standardized triage scenarios (survey design) to compare 3 groups [1. no decision support, 2. deep learning support, 3. interpretable model support]. Multiple clinician types (trauma surgeons, emergency medicine physicians, communications nurses) are surveyed and rate the support models with respect to overall acceptability, preference, and confidence in model output. An additional trial demonstrates the feasibility of model implementation and generates preliminary data for a larger trial. Using a randomized design in which triage personnel are presented with either no decision support or model support, the accuracy of trauma activation levels with and without decision support are compared and balanced against clinician feedback regarding the model and other characteristics (e.g., acceptability, ease of use). Implementation feasibility is demonstrated when the chosen model improves trauma activation accuracy, decreases variability, and is considered acceptable for use in the clinical setting.

Working with survey design experts, 12 standardized triage scenarios are developed representing a range of patient presentations that vary with respect to injury severity, presence/absence of prehospital information, and whether or not other information is missing (Table 5).

TABLE 5
Standardized Scenarios for Assignment
of Trauma Activation Level

| | Injury Severity | Prehospital Report | Other Missing |
|---|---|---|---|
| | (Mild/Moderate/ | Available | Information |
| | Severe) | (Yes/No) | (Yes/No) |
| 1 | Mild | Yes | No |
| 2 | Moderate | Yes | No |
| 3 | Severe | Yes | No |
| 4 | Mild | No | No |
| 5 | Moderate | No | No |
| 6 | Severe | No | No |
| 7 | Mild | Yes | Yes |
| 8 | Moderate | Yes | Yes |
| 9 | Severe | Yes | Yes |
| 10 | Mild | No | Yes |
| 11 | Moderate | No | Yes |
| 12 | Severe | No | Yes |

A randomized survey is performed using the standardized triage scenarios developed herein. Three groups are compared (1. No decision support, 2. Deep learning support, 3. Interpretable model support) amongst multiple clinician types (e.g., trauma surgeons, emergency medicine physicians, communications nurses). Clinicians rate the support models with respect to overall acceptability, preference, and confidence in model output. An ideal model is acceptable to and preferred by the end-user with a high level of confidence in the model output.

Additional data identified opportunities for process/quality improvement in the existing trauma activation workflow that can be leveraged to aid implementation. A trial demonstrates the feasibility of model implementation and generates data for a larger prospective trial. A randomized design in which triage personnel are presented with either no decision support or model support is created. Accuracy of trauma activation levels with and without decision support is compared and balanced against clinician feedback regarding the support and other characteristics (e.g., acceptability, ease of use). Implementation is deemed feasible when the chosen model improves trauma activation accuracy, decreases variability, and is considered acceptable for use in the clinical setting.

Neither Cribari nor NFTI criteria can be used for prospective predictions. In this work, the study is not using Cribari or NFTI in a prospective manner to assess patients. Rather, the study is asking the algorithm to assess patients using its internal criteria, and then it checks its work (much like is done for qualitative review) against NFTI and Cribari. As an example, the algorithm predicts a specific trauma team activation level for one patient based upon a specific variable, then checks its prediction against NFTI. If that patient was not correctly triaged, the algorithm will adjust the “weight” of that variable to mark it as less important in future iterations. This training process is performed repeatedly until the decision thresholds are optimized (as much as possible) and available pre-hospital predictor variables have been weighted for importance in prediction. These variables, weights, and decision thresholds are then used in a prospective manner to predict the correct level of trauma team activation.

### Example 5: Demonstrable Improvement at Triage Decision-Making Performance with a Machine Learning Model

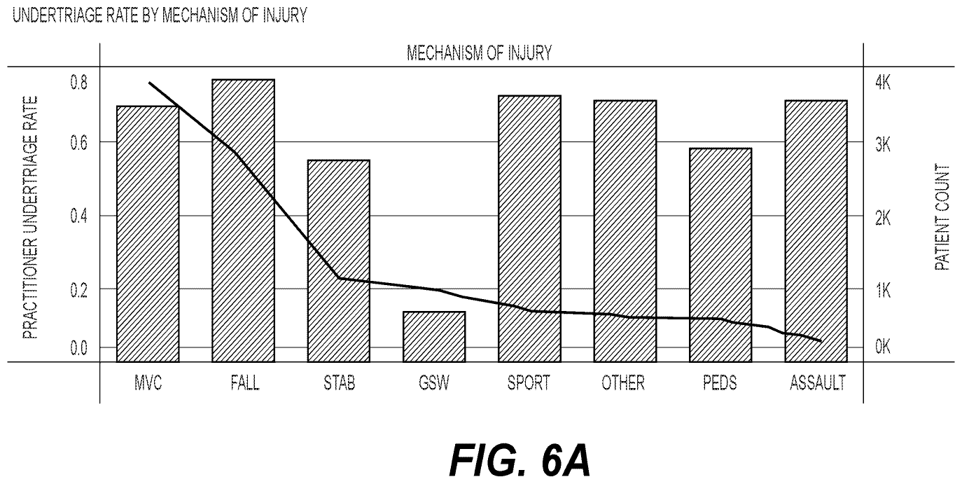

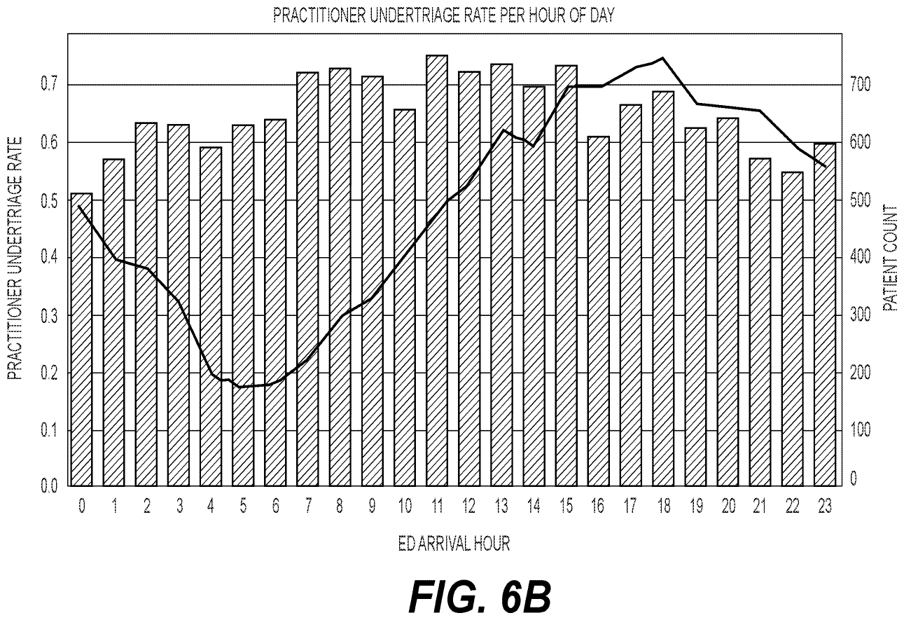

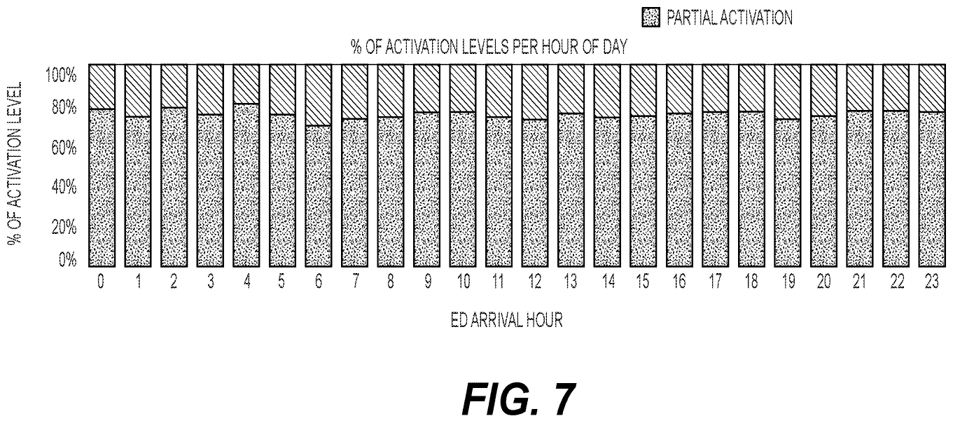

The study investigated the variation in undertriage rate by ED staff for different patient arrival times, as shown in FIG. 6, Panel B. The study showed that undertriage rates appeared inconsistent for different arrival times, despite ground truth activation levels being roughly constant throughout the day as shown in FIG. 7. A two-sample proportion test comparing undertriage rates in the ED in the daytime (6 am to 6 μm) vs nighttime (6 μm to 6 am) showed a significant difference at a significance level of α=0.05 (two-sided test) with undertriage being significantly higher in the daytime.

In order to determine where classification errors occur in the ED, the study considered under and overtriage rates for different groups within the data. The study performed tests at the α=0.05 significance level to determine if differences in these rates for different groups were significant. The study kept any discovered discrepancies in mind as the study evaluated the performance of the model.

In the data, the ED staff had an undertriage rate of 65.5% and overtriage rate of 16.5%. Rates for under/overtriage goals and current ED staff levels are presented for reference in Table 6 below.

TABLE 6
Definitions, goals and ED staff rates for undertriage and overtriage.

| | Full Activation (1) | Partial Activation (0) |
|---|---|---|
| Description | Patient needs most | Patient needs less |
| | intensive care | intensive care |
| Associated Error | Undertriage (false | Overtriage (false |
| | negative) | positive) |
| Desired Error Rate | 5% | 25-35% |
| (Institutional Standard) | | |
| ED Staff Error Rate | 65.5% | 16.5% |

The mechanisms of injury were separated into eight categories (FIG. 6, Panel A):
Motor vehicle collision (MVC)
Injury due to fall (FALL)
Stab wound (STAB)
Gunshot wound (GSW)
Injury due to sports (SPORT)
Pedestrian struck by vehicle (PEDS)
Injury due to assault (ASSAULT)
Any injury not related to the above categories (OTHER)

One-versus-rest two-sided proportion testing at the α=0.05 significance level revealed a significant difference in the undertriage rate for six of eight injury mechanisms, listed here along with whether associated undertriage rates were higher or lower than the rest of the mechanisms grouped together:
1) Gunshot wounds-lower undertriage rate
2) Fall-higher undertriage rate
3) Motor vehicle collision-lower undertriage rate
4) Pedestrian struck by vehicle-lower undertriage rate
5) Sports injuries-higher undertriage rate
6) Stab wounds-lower undertriage rate

Similar tests comparing overtriage rates for different mechanisms of injury showed that each overtriage rate was significantly different from the rest of the mechanisms. Considering the differences in undertriage rates for different injury mechanisms, this is not surprising, as undertriage and overtriage are generally inversely related.

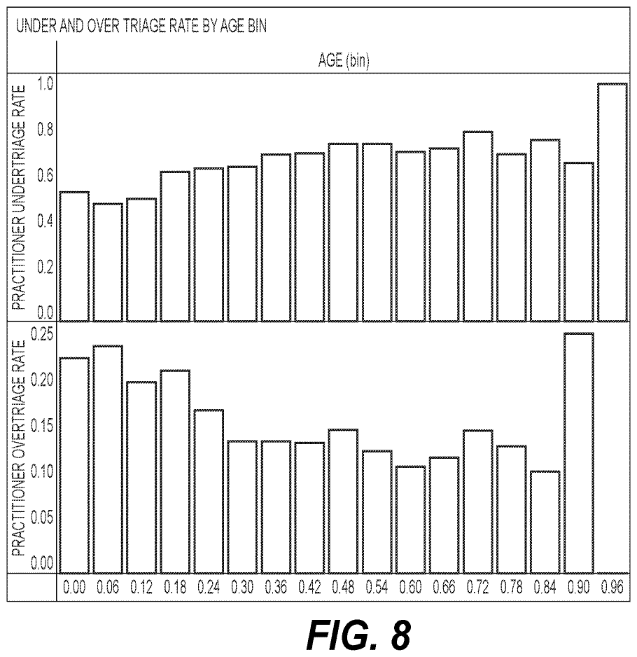

Patient age was another feature that had an effect on under and overtriage rates in the ED. FIG. 8 shows the under and overtriage rates for patients of different ages. These plots show a general trend of undertriage rates generally decreasing as age increases and overtriage rates generally increasing as age increases.

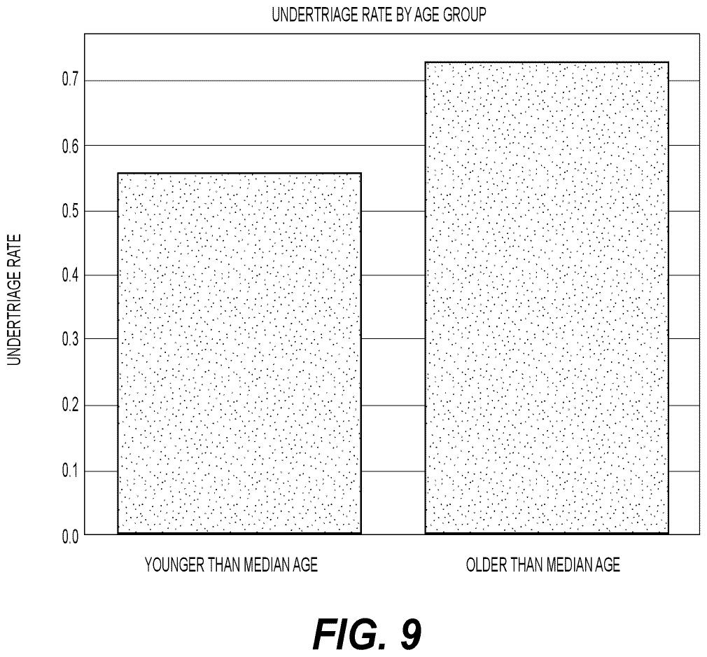

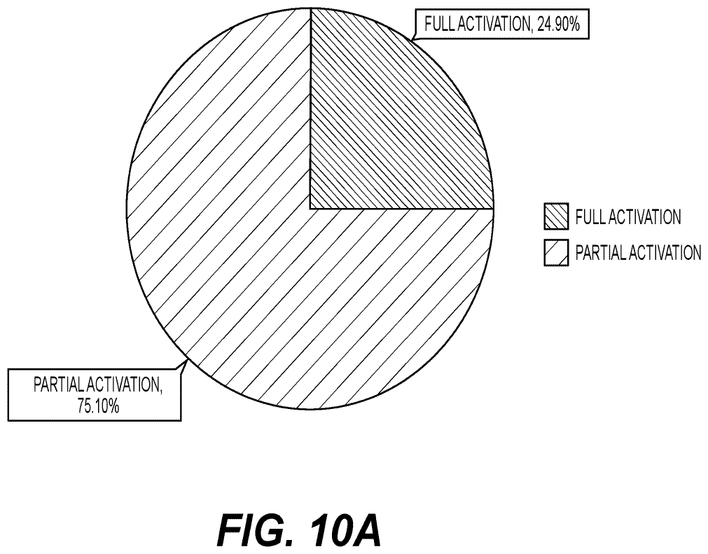

A two-sample test comparing undertriage rates for patients above and below the median age in the dataset of 47.57 years showed a significantly greater rate of undertriage for patients above the median age (FIG. 9).

Data contains 10959 records from patients admitted to the ED from 2014 to 2021. There are over 200 features in the raw data that are related to the patient's health history and pre-hospital treatments. These features include patient demographic information, mechanism of injury, pre-hospital interventions, comorbidities of patients, and ED procedures performed for patients, along with many other variables.

The data includes two ground truth variables for activation level: Cribari and NFTI. They show the level of care the patient needs. The ground truth variables are determined after the patient's treatment using different criteria. The Cribari variable is available in all records in the data whereas the NFTI variable is not available in records from 2014 to 2019.

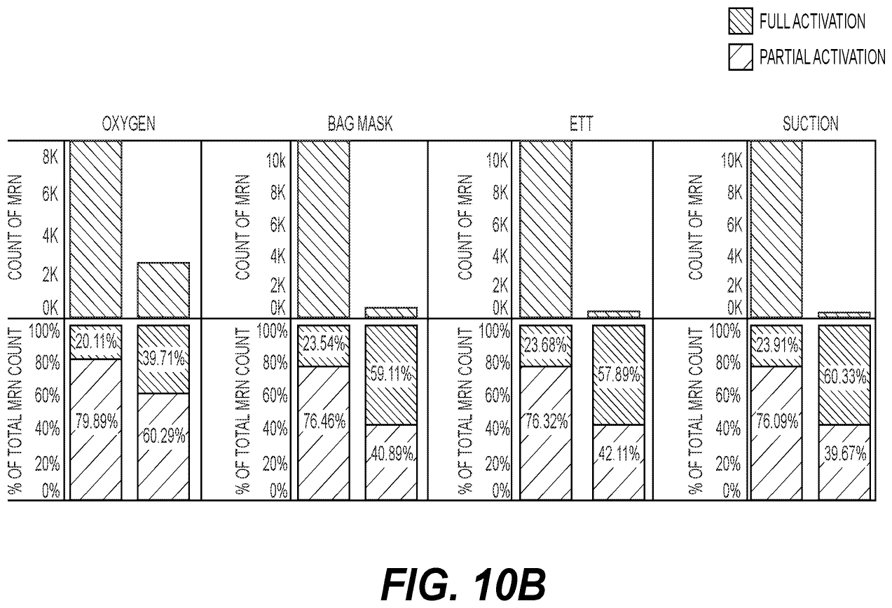

Both ground truth variable is binary. ‘1’ is ‘Full Activation’ which means the patient needs a high level of care. ‘0’ is ‘Partial Activation’ which means the patient does not need a high level of care (see FIG. 10, Panel A).

For features with missing data that were not dropped, the study saw that there were several sets of co-occurring missing values seen in individual samples. The features that had co-occurring missing values were related to vital sign measurements, and each set of missing features was made up of different summary values of the same set of values. Because missing feature values occurred together in these groups, and because the missing data was not missing at random, but instead due to underlying circumstances regarding the patient, the study used the MICE (Multivariate Imputation By Chained Equations) algorithm to impute missing values. This algorithm imputes missing values at random, then iteratively updates the imputed values using a coherence measure for the entire dataset as an optimization metric. Once the missing values have been imputed, the data imputer can be saved for future use.

Feature selection is an integral step in model building as it helps to reduce the dimensionality of the data, mitigate overfitting, and improve the model's performance. In this study, the Recursive Feature Elimination (RFE) technique was employed for the feature selection task.

The RFE method iteratively removes the least important features from the dataset based on the importance ranking assigned by the estimator. The algorithm continues until the desired number of features is reached or the performance metric reaches a satisfactory level.

The study utilized the X GBooster and Random Forest algorithms as the estimators in the RFE method. X GBooster is a powerful machine learning algorithm that is widely used for classification and regression problems. It has gained popularity due to its ability to handle missing values, scale to large datasets, and provide excellent accuracy.

Furthermore, to evaluate the efficacy of the Recursive Feature Elimination method with X GBooster as the estimator, the study also employed the Random Forest algorithm as an alternate estimator for feature selection which is a versatile machine learning algorithm that is suitable for both regression and classification problems and is known for its robustness against noise and outliers.

As can be seen in FIG. 10, Panel B, the study analyzed the distribution of the ground truth variable for each value of features selected. The features are as follows: O₂ (Oxygen), BVM (Bag-valve mask ventilation), ETT (Intubation), and Suck (Airway suction). For example, the percentage of full-activation patients is larger in a set of samples that received O₂. If the study observes the features as ‘1’ in the patient, the patient is likely to need full activation as stated in FIG. 10, Panel B.

If the model is to be used as a support tool for practitioners, they need to be able to trust it, understand how confident it is in each prediction it makes, and be able to check how different features of a given sample (i.e. attributes of an incoming patient) influence the decision being made. In order to satisfy these needs, the study implemented the LIM E (Local Interpretable Model-agnostic Explanations) explainer with the model [M. Ribeiro and S. Singh and C. Guestrin. “Why Should I Trust You?”: Explaining the Predictions of Any Classifier. Proceedings of the 22^{nd} A CM SIGK DD International Conference on Knowledge Discovery and Data Mining, San Francisco, CA, USA, Aug. 13-17, 2016].

The LIME explainer works by taking in a single input (a single patient's data) and performing small perturbations around the feature values to see how changes to these values would affect the output classification. The explainer does not provide a general explanation for how the model makes predictions—it only gives information about how individual classifications were decided. This is suitable for these needs because if it is implemented as a support tool it will only need to explain one decision at a time.

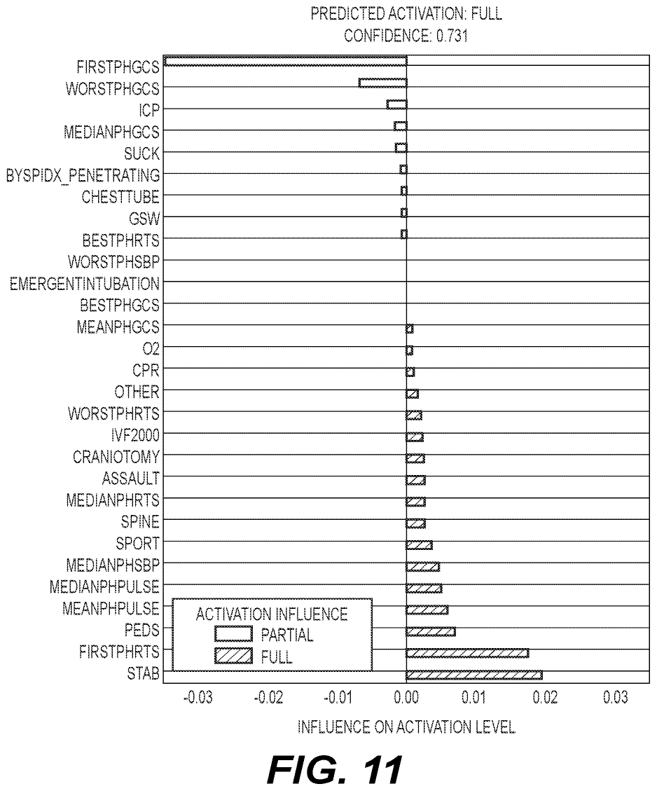

A visualization based on the output of the LIM E explainer is shown in FIG. 11. The activation level decided by the model is shown at the top of the figure, with a confidence level in the classification reported below it. The confidence level is taken as probability assigned to the selected activation level by the model. The bar plot in the figure has one bar per feature, sorted by influence (largest influence toward partial activation at the top and largest influence toward full activation at the bottom). The color of each bar shows whether the value of the feature it represents influenced the decision of the model toward full or partial activation, and the size of the bar shows how strong that influence was. The example shown in the figure is a patient classified by the model as needing full activation, with the feature that influenced the decision in the direction of full activation the most being that it was a stab wound (“STAB”).

The overall accuracy, undertriage rate, and overtriage rate of the model were all significant improvements over the classification performance of ED staff, according to a series of two-sample proportion tests at the α=0.05 significance level. Performance metrics for the model and for the ED staff are presented in Table 7 below.

TABLE 7
Comparison of undertriage rates, overtriage rates,
and overall accuracy for triage level classification
by the model compared to practitioners in the ED.

| | | ED Staff | |
|---|---|---|---|
| | Ensemble Model vs. | Classification vs. | |
| | Cribari Ground | Cribari Ground | Better |
| | Truth | Truth | Predictor |
| Accuracy | 90.3% | 71.3% | Model |
| Undertriage Rate | 23.3% | 65.5% | Model |
| (false negative rate) | | | |
| Overtriage Rate | 5.18% | 16.5% | Model |
| (false positive rate) | | | |

Comparing multiple metrics with Cribari method as the ground truth, shows that the model performs better than human classification in terms of accuracy as well as the false positive and false negative error rates. The success criteria with over-triage error rate of <5% and under-triage error rate of 25-35% is also almost achieved, considering the fact that the model utilized the pre-treatment features only, and thus can be replicated with least effort in any trauma center. This is in contrast with the Cribari ground truth, which took into consideration the combination of pre and post treatment features for modeling.

### Example 6: Machine Learning Improves the Accuracy of Trauma Team Activation Level Assignments in Pediatric Patients

The study retrospectively collected data from the institutional trauma registry and electronic medical record at a Pediatric Trauma Center for all patients (age <18 y) who triggered a trauma team activation (1/2014-12/2021), including: demographics, mechanisms of injury, comorbidities, pre-hospital interventions, numeric variables, and the six “Need for Trauma Intervention (NFTI)” criteria. Three machine learning models (Logistic Regression, Random Forest, Support Vector Machine) were tested 1000 times in separate trials using the union of the Cribari and NFTI metrics as ground-truth (Injury Severity Score >15 or positive for any of 6 NFTI criteria ¼ full activation). Model performance was quantified and compared to emergency department (ED) staff.

To mimic the information available to ED staff at or near the time of patient arrival, predictive variables, or features, were identified based on availability either in the pre-hospital setting or immediately after patient arrival. Multiple features were extracted from International Classification of Diseases (ICD) 9/10 codes, pre-hospital interventions, and comorbidity text data and encoded into variables. Additional features derived from the original dataset were engineered based on the most up-to-date institutional trauma center triage guidelines. These features were then used as inputs into the machine learning algorithm (see Table 4 above for exemplary predictors utilized by the machine learning model).

Table 8 below shows the top 10 features for each of the models: logistic regression, random forest, and the support vector machine model. Bold features designate those that are shared between all models and underlined features designate those that are shared between two of the three models.

TABLE 8

| Feature | | | Support Vector |
|---|---|---|---|
| Importance | Logistic regression | Random Forest | Machine |
| 1 | Blood Transfusion | GCS <8 | Blood Transfusion |
| 2 | Gunshot Wound - Head | Mean Pre-Hospital | Gunshot Wound - |
| | | Glasgow Coma Scale | Torso |
| 3 | Gunshot Wound - | Mean Pre-Hospital | Intraosseous |
| | Torso | Revised Trauma | Access |
| | | Scale | |
| 4 | Intubation | Blood Transfusion | Gunshot Wound - |
| | | | Head |
| 5 | Bleeding Disorder | Multiple Fractures | GCS <8 |
| 6 | Intraosseous Access | Airway Management | Significant Head |
| | | | Injury |
| 7 | Significant Head Injury | Oxygen | Airway Suction |
| 8 | Airway Suction | Intubation | Bleeding Disorder |
| 9 | CPR | Intravenous Fluids | Major Psych |
| | | | Diagnoses |
| 10 | GCS <8 | Significant Head | Pelvic Fracture |
| | | Injury | |

Missing values were identified for pre-hospital SBP, GCS, and RTS. In order to exploit the relationships between each of the clinical variables, the study utilized multivariate iterative imputation with chained equations (MICE) [Getz K, Hubbard R A, Linn K A. Performance of multiple imputation using modern machine learning methods in electronic Health records data. Epidemiology 2023; 34:206e15]. MICE is the most widely utilized imputation method for clinical data and works by iteratively learning how to impute a given variable, using the other variables, until convergence.

The primary outcome of interest was predicted trauma activation level, defined as the “level” assigned to the patient at the time of arrival to the ED. In this exemplary embodiment, three levels of resource activation are possible: Level 1 (full trauma activation), Level 2 (partial trauma activation), and trauma consult (partial trauma activation). Each patient is assigned an actual activation level by ED staff at the time of their arrival. However, because of the potential for over-/under-triage due to innate human bias and error, this actual activation level cannot be used as the ground-truth for predictive modeling.

Therefore, the study chose to use the union of two retrospective assessment tools: 1) the Cribari matrix and 2) the NFTI criteria. To assess over- and under-triage, a predicted activation level was assigned to each patient where a full activation was triggered by either ISS >15 or a positive response to any of the six NFTI criteria. If all six NFTI criteria were negative and the ISS <15, a partial activation was predicted. The predicted activation level was then compared to the actual activation level for each patient to determine over-vs. correct vs. under-triage. This approach is also referred to in the application as a “combination of ISS and NFTI.”

Three common, state-of-the-art, explainable classical machine learning models were trained: Logistic Regression, Random Forest, and Support Vector Machine. Each model was trained on all 104 features, including patient characteristics, comorbidities, and outcomes. Each of the models were hyperparameter tuned according to a grid-search so that the best version of each model was compared to ED staff. Labels for training data were created using the union of the Cribari+NFTI ground-truth.

To train the prediction models, the dataset was randomly divided into training (80%), and test (20%) data. For example, in the training set, 209 patients received an activation level of 1 (full), while 1015 patients received an activation level of 0 (partial/consult). In the testing set, 41 patients received an activation level of 1 (full), while 101 patients received an activation level of 0 (partial/consult). All analyses were performed using Python 3.7.0 and the models were trained and evaluated with the Scikit-Learn 1.0.1 package.

Logistic regression learns a linear combination of the input variables, which is then scaled to probabilities within the final logistic function. By nature, this type of machine learning is interpretable and explainable, in that the learned linear coefficients can be used to understand which of the clinical variables most heavily influenced the final model prediction. For reproducibility, the selected parameters were: balanced class weights, 2e³ max iterations, C=10, and the Newton-CG solver.

Random Forest is an ensemble-based machine learning model that learns a number of simple decision trees on sub-samples of the dataset and uses averaging to combine the decision tree results to improve accuracy and prevent overfitting. Due to its decision tree backbone, the study can explicitly understand which binary decisions were made and are most important to the ultimate prediction. For reproducibility, the selected parameters were: balanced class weights, 1e³ max estimators, 5 max depth, entropy criterion, and square root to determine max features.

Support vector machines find a boundary between two binary classes that allows for separation by maximizing the distance between this boundary and the data points. For this model, the linear support vector machine was used, in which a linear boundary is learned and allows for straightforward interpretability, as the user can determine which of the clinical variable's coefficients are most heavily weighted in constructing the linear boundary. For reproducibility, the selected parameters were: balanced class weights, linear kernel, 1e⁸ max iterations, and C=0.95.

The study utilized the area under the receiver operating characteristic curve (AUC) and F1 score as the primary performance metrics because they are typically used to evaluate the predictive strength of binary classification models in the setting of imbalanced data [Jeni L A, Cohn J F, De La Torre F. Facing imbalanced data-recommendations for the use of performance metrics. In: 2013 humane association conference on affective computing and intelligent interaction; 2013. p. 245e51.]. AUC considers the entire range of classification thresholds for a model prediction's probability and thus captures the model's ability to correctly classify both positive and negative instances, regardless of imbalance. F1 evaluates models that strike a good balance between correctly identifying positive instances while minimizing false positives, thus providing a more balanced metric that highlights model performance on the minority class. Both of these metrics have a 0-1 range, where 1 is best and 0 is worst. Accuracy, in comparison, is a poor evaluation metric given imbalanced data due to its insensitivity to the minority class and the potential for high accuracy driven solely by the majority class. However, for the sake of completeness, the accuracy metric is also reported. Resampling (with different random initializations), retraining, and retesting each model 1000 times created 95% confidence intervals. Univariate analyses were performed using MATLAB (R2022b, The MathWorks Inc, Natick, MA).

There were 1366 patients included in this analysis. For full patient demographics and injury characteristics. The mean±standard deviation (SD) ISS was 9.39±10.1 [median (IQR)=6.00 (1.0e13.0)] in the overall group and 18.01% (n=246) had an ISS >15. Overall, 26.65% (n=364) were positive for at least one of the NFTI criteria. ED staff triggered a full trauma team activation (actual activation level) in 16.84% (n=230) of patients.

Using the union of the Cribari+NFTI metrics as ground-truth, ED Staff had an accuracy of 75%, but an AUC of 0.73±0.04 and an F1 score of only 0.485 (Table 9 below (Machine learning performance metrics compared to ED staff with Cribari+NFTI as ground-truth.))

TABLE 9

| | Logistic | Random | Support Vector | ED |
|---|---|---|---|---|
| | regression | Forest | Machine | Staff |
| AUC ± | 0.786 ± | 0.783 ± | 0.813 ± | 0.730 ± |
| 95% CI | 3.33e⁻¹⁶ | 0.003 | 4.12e⁻⁵ | 0.037 |
| F1 Score ± | 0.779 ± | 0.791 ± | 0.800 ± | 0.485 |
| 95% CI | 2.22e⁻¹⁶ | 0.006 | 3.33e⁻¹⁶ | |
| Accuracy ± | 0.782 ± | 0.804 ± | 0.803 ± | 0.750 |
| 95% CI | 1.11e⁻¹⁶ | 0.005 | 2.22e⁻¹⁶ | |

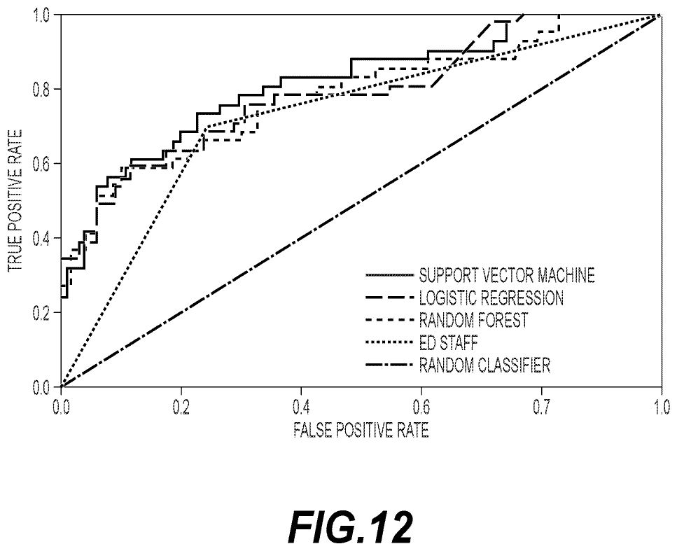

All machine learning models outperformed ED staff in all performance metrics. Of the three machine learning models tested, the support vector machine model had the best performance, with the highest AUC of 0.813±4.12e⁻⁵ and an F1 Score of 0.800±3.33e⁻¹⁶ and the lowest variance (95% CI). The associated receiver operating characteristic curves are illustrated in FIG. 12.

Over- and under-triage also occur in model predictions. Therefore, it is imperative to understand the rates of over- and undertriage and to compare them to over- and under-triage by ED staff. As mentioned above, ED staff had an overall over-triage rate of 5.05% (n=69) and an under-triage rate of 19.99% (n=273). Over and under-triage rates for all three machine learning models and for ED staff using the same test dataset (that excludes model training data) are shown in Table 10 below (proportion of patients over- and under-triaged using the testing dataset. Over-triage rates were calculated as the number of patients predicted to require a full activation by each model, but did not require a full activation according to the Cribari+NFTI ground-truth, divided by the total number of patients in the testing set (n+142). Similarly, under-triage rates were calculated as the number of patients predicted to not require a full activation by each model, but did require a full activation according to the Cribari+NFTI ground-truth, divided by the total number of patients in the testing set).

TABLE 10

| | Logistic | | Random | Support Vector | | ED | |
|---|---|---|---|---|---|---|---|
| | regression | | Forest | Machine | | Staff | |
| Over-triage (%, n) | 9.86% | (14/142) | 5.63% (8/142) | 8.45% | (12/142) | 7.04% | (10/142) |
| Under-triage (% , n) | 12% | (17/142) | 14.08% (20/142) | 11.27% | (16/142) | 21.13% | (30/142) |

The support vector machine model minimized under-triage with a rate of 11.27% (n=16), while maintaining over-triage at a similar rate of 8.45% (n=12).

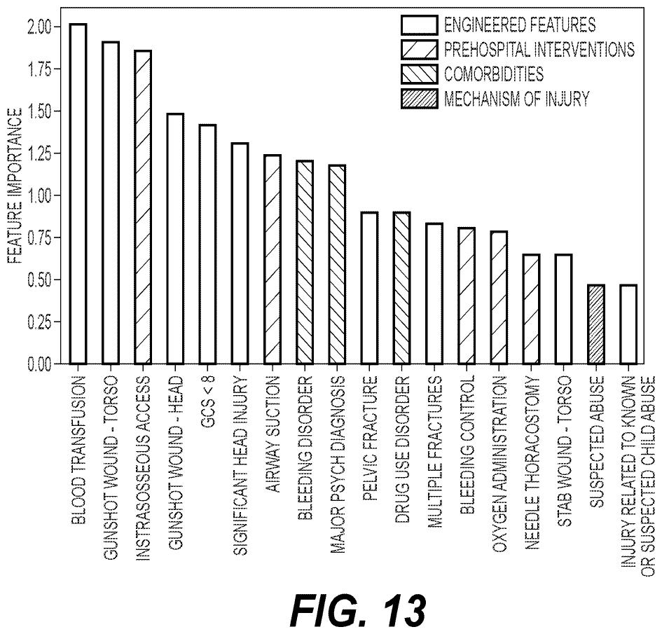

The top five features in terms of importance in decision-making for the support vector machine model were: (1) blood transfusion (i.e., patient receiving or has received blood transfusion prior to arrival); (2) gunshot wound to the torso; (3) pre-hospital intraosseous access; (4) gunshot wound to the head; and (5) Glasgow Coma Scale score <8 (FIG. 13).

All machine learning models outperformed ED staff in all performance metrics. These results show that data-driven methods can optimize trauma team activations in the ED, with improvements in both patient safety and hospital resource utilization.

ED staff had 75% accuracy, an area under the curve (AUC) of 0.73±0.04, and an F1 score of 0.49. The best performing of all machine learning models, the support vector machine, had 80% accuracy, AUC 0.81±4.1e⁻⁵, F1 Score 0.80, with less variance compared to other models and ED staff.

The results of this study demonstrate that machine learning based tools can be used to both improve accuracy and decrease variability associated with trauma team activation decisions in the ED. Specifically, the support vector machine model used in this study improved the AUC by 8.3% and decreased variability by several orders of magnitude. These performance improvements were obtained while reducing under-triage and maintaining overtriage rates, using a fully explainable model. Overall, these results show that data-driven methods can be used to optimize trauma team activations in the ED, which leads to improvements in both patient safety and hospital resource utilization.

Interestingly, of the ten features most heavily weighted by the model, six were engineered features derived from the institutional triage criteria, showing that the basis for the model's activation level predictions aligns with that used by healthcare providers. Further, pre-hospital interventions that may imply a patient has more severe injuries (i.e., intraosseous access, airway suction, bleeding control, oxygen administration, and needle thoracostomy) accounted for five of the top 15 features (FIG. 13).

During the training process, each time the algorithm checks its work against the ground-truth, it adjusts this weighting. In certain embodiments, ablation studies serially eliminate individual variables to determine their impact on prediction accuracy. This allows elimination of selected variables with minimal impact and, ultimately, arrives at ten or fewer predictor variables that can be used to accurately assign a trauma team activation level.

### Example 7: To Prioritize Patient Safety, Cribari+NFTI Appears Best for Training a Machine Learning Algorithm to Predict Trauma Team Activation Level

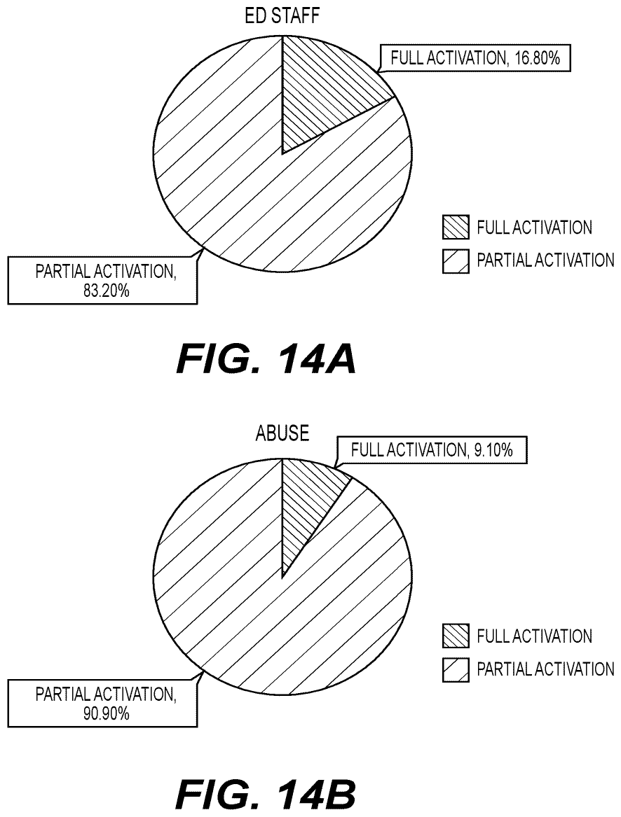

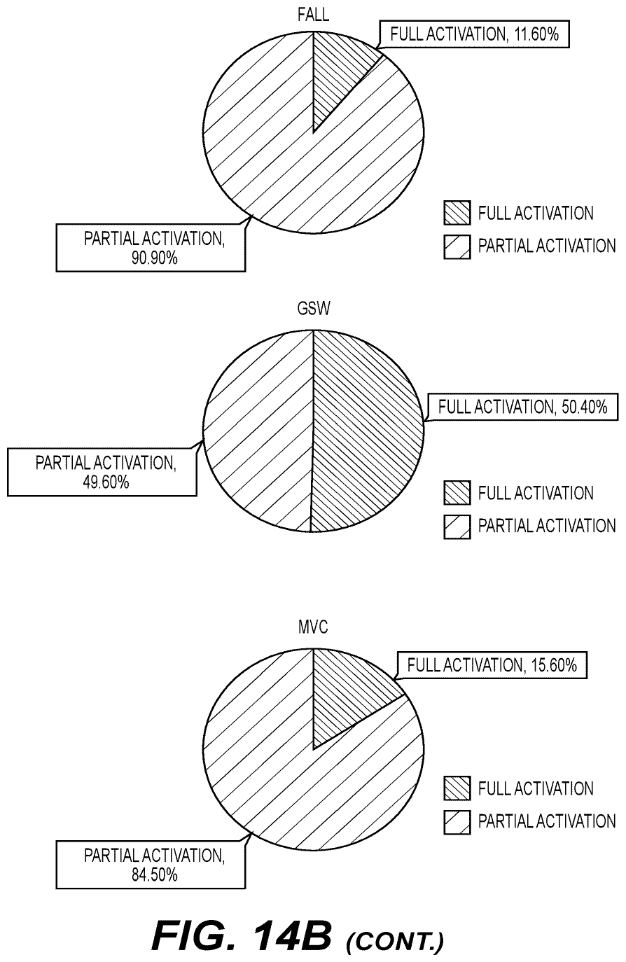

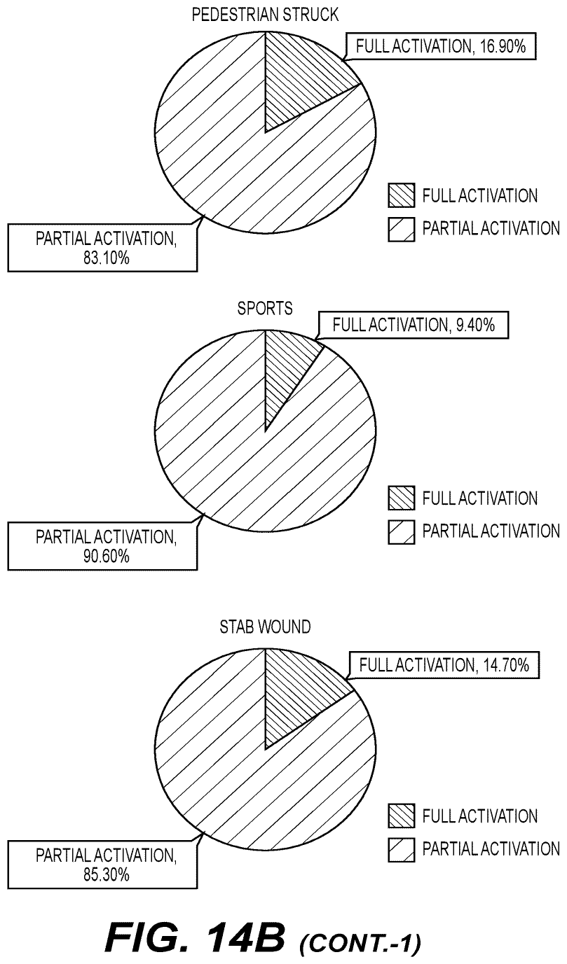

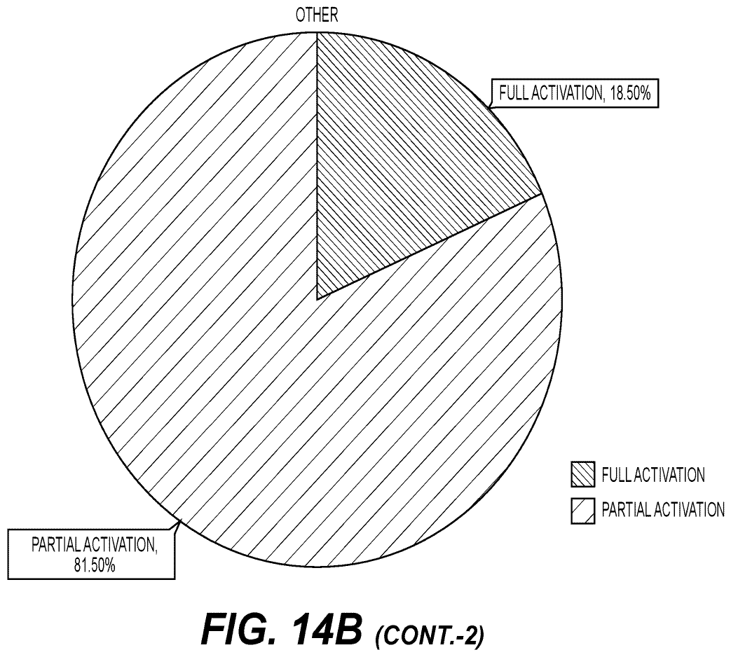

Of the 1,366 patients, trained ED staff triggered a full activation in 230 (16.84%) patients and a partial activation in 1,136 patients (83.16%; FIG. 14, Panel A). Cribari indicated full activation in 246 (18.01%) and partial activation in 1,120 (81.99%). NFTI indicated full activation in 364 (26.65%) and partial activation in 1,002 (73.35%). NFTI+Cribari indicated full activation in 434 (31.77%) and partial in 932 (68.23%). The breakdown of full and partial activations for each mechanism of injury is shown in FIG. 14, Panel B.

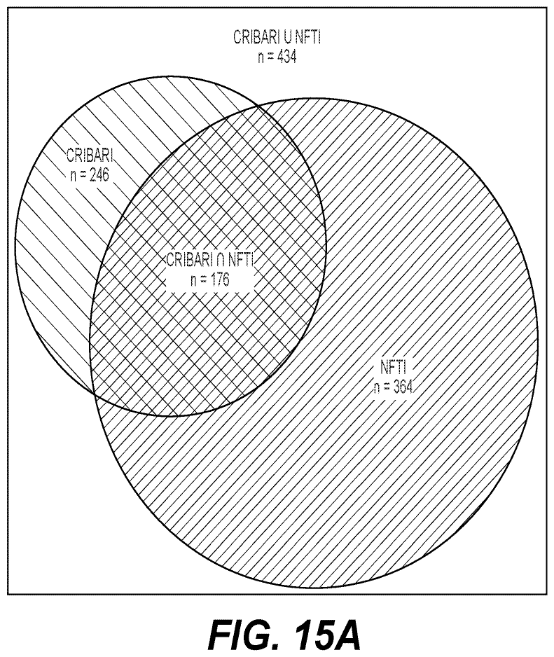

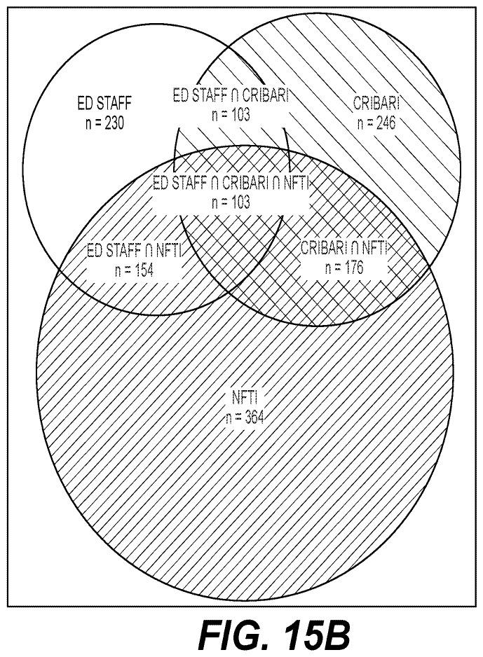

Cribari and NFTI identified incompletely overlapping sets of patients predicted to require a full activation (FIG. 15, Panel A). There was complete agreement between Cribari, NFTI, and ED staff in only 7.03% (n=96) of patients (FIG. 15, Panel B), but when the combined Cribari+NFTI metric was used there was agreement with ED staff in 11.79% (n=161) of patients. To better understand the behavior of the three ground-truths, patient characteristics were compared among the three groups for under-triage and over-triage.

### Under-Triage

There were 143 (10.47%) patients who were considered under-triaged using Cribari in comparison to 210 (15.37%) patients using NFTI. Combined, the Cribari+NFTI metric produced an under-triage rate of 19.99% (n=273). There were no differences in demographics between patients who were under-triaged according to each of the three ground-truths. NFTI and Cribari+NFTI were more sensitive to under-triage in patients with penetrating mechanisms of injury (p=0.006) and detected more under-triaged children with stab wounds compared to Cribari (p=0.014). There were no differences between the groups for prehospital interventions and vital signs upon arrival to the ED.

### Stab Wounds

There were 95 patients stabbed in the overall cohort, with a mean ISS of 4.96 (7.05). Fourteen patients (14.74%) with stab wounds triggered a full activation by ED staff. Cribari indicated that only 8 patients (8.42%) required a full activation, while NFTI and Cribari+NFTI deemed that 32 (33.68%) and 34 (35.79%) required a full activation, respectively. Of the 34 patients identified by the Cribari+NFTI metric as requiring a full activation, 8 (23.53%) had an ISS >15, 8 (23.53%) were transfused within 4 hours of arrival, 4 (11.76%) were mechanically ventilated outside of procedural anesthesia within 3 days of arrival, 23 (67.65%) went directly from the ED to the operating room within 90 minutes of arrival, 3 (8.82%) went directly to interventional radiology from the ED, 6 (17.85%) went from the ED to the ICU with an ICU LOS of 3 or more days, and 2 (5.88%) died within 60 hours of arrival. Patients with stab wounds who were deemed undertriaged by NFTI (n=24, 25.26%) had a mean ISS of 6.2 (8.6), explaining why these patients were missed by Cribari. However, 75% of the NFTI under-triaged stab wounds (18/24) went directly from the emergency department to the operating room.

### Over-Triage

There were 126 patients who were considered over-triaged using Cribari (9.22%), while 75 patients were considered over-triaged using NFTI (5.49%). The combined Cribari+NFTI metric produced an over-triage rate of 4.98% (n=68). There were no differences in demographics for patients who were over-triaged according to each of the three ground-truths. Over-triage was particularly common for gunshot wounds, regardless of ground-truth. Compared to NFTI and Cribari+NFTI, Cribari indicated over-triage in more patients with abuse as mechanism of injury (p<0.001), more patients that required prehospital airway management (p<0.001), and more patients that underwent CPR in the prehospital setting (p=0.017). In addition, compared to NFTI and Cribari+NFTI, Cribari indicated over-triage in patients with mean lower GCS scores in the ED (p<0.001).

### Outcomes

Overall, the mortality rate was 3.37% (n=46) in this dataset. Of the patients who died, 35 (76.09%) triggered a full activation by trained ED staff, 32 (69.57%) required a full activation according to Cribari, and 46 (100.00%) required a full activation according to both NFTI and Cribari+NFTI (p<0.001). Of the 14 patients “missed” by the Cribari metric, 7 had no ISS recorded in the trauma registry, 5 were recorded as a death upon arrival or death in the ED, an additional 3 patients went directly from the ED to the operating room, and 6 patients were admitted directly to the ICU. For patients who were under-triaged, there were no significant differences between the three ground-truths for mortality (p=0.803). However, the mortality rate was significantly higher in the Cribari over-triage group (7.14%, n=9), compared to NFTI and Cribari+NFTI (0.00%, n=0, p=0.005). Mean length of stay for the overall dataset was 4.26 (7.76) days. For patients who were under-triaged, there were no significant differences between the three ground-truths for LOS (p=0.664). However, Cribari indicated over-triage in patients with significantly longer LOS (p=0.017), compared to NFTI and Cribari+NFTI.

The study demonstrated that the NFTI and Cribari+NFTI metrics were more sensitive to penetrating mechanisms of injury and specifically detected more under-triaged children with stab wounds compared to Cribari. This is the first time this has been demonstrated. This is the first study to demonstrate a difference in detection of particular injury mechanisms. The difference in detection of stab wounds in the population of study is significant, as the Cribari metric uses a minimum threshold of ISS >15 to indicate a full trauma team activation is required, and the mean ISS of patients with stab wounds, who often have limited bodily regions involved, was only 4.95±7.05. Patients with stab wounds are illustrative of why the Cribari+NFTI metric is better than either metric alone from a patient-safety perspective.

For stab wounds specifically, approximately 68% of the patients identified as requiring a full activation by the combination metric were taken directly from the ED to the operating room within 90 minutes of arrival, indicating a significant need for resources available at the time of arrival. While the NFTI metric alone captured most of the patients requiring a full trauma team activation and all of the patients that went directly to the OR, the Cribari metric captured an additional 2 patients with stab wounds and with an elevated ISS.

The characteristics of patients who were over-triaged also illustrate the limitations of the Cribari metric, in particular. These results demonstrate that Cribari frequently indicated over-triage in patients with potentially severe injuries, as more patients that required prehospital airway management or CPR and more patients with mean lower GCS scores in the ED were over-triaged according to the Cribari metric.

Perhaps most importantly, the Cribari+NFTI and NFTI metrics captured 100% of the mortality in this dataset, while the Cribari metric only captured 69.6%. In this study's comparison of metrics, one of the NFTI criteria includes mortality within 60 hours of arrival, thus leading to an almost 100% sensitivity for this outcome. However, from a patient safety perspective, it is concerning to note that the mortality rate was significantly higher in the Cribari over-triage group (7.14%) compared to that of the NFTI and Cribari+NFTI metrics (0%).

One concern in relation to use of these metrics is that each was created for the determination of triage quality in a retrospective fashion, and thus cannot be accurately utilized in pre-hospital or hospital triage settings. However, when used as ground-truth(s) for a predictive model, these metrics can be used to train the model to identify and weight the most predictive pre-hospital variables and, thus, for prospective hospital triage (Liu C W, Chacon M, Crawford L, Polydore H, Ting T, Wilson N A. Machine Learning Improves the Accuracy of Trauma Team Activation Level Assignments in Pediatric Patients. J Pediatr Surg. 2023 September 22: S0022-3468(23)00551-1). Therefore, the study has prioritized sensitivity toward patient safety as opposed to conservation of hospital resources, and, finds the Cribari+NFTI metric to be an ideal ground-truth for training predictive models.

The difficulty of determining an appropriate ground-truth for training a machine learning algorithm makes this work highly significant. Furthermore, pediatric trauma systems have essentially grown out of adult trauma systems, and the pediatric literature is limited with respect to exploration of how these metrics behave specifically for pediatric patients. In addition to the usefulness for predictive modeling, this exploration of an appropriate ground-truth has led to tangible quality improvement efforts in clinical practice and can provide similar benefit for other pediatric trauma programs.

While various embodiments have been described above, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

1. A method of training a machine learning model to predict the assignment of medical resources required to treat a patient for traumatic injury, the method comprising:
(a) establishing a ground-truth, wherein the ground-truth corresponds to a standardized trauma activation level for a standardized patient with a standardized set of clinical conditions;
(b) assigning a first trauma activation level for an individual patient identified with a plurality of clinical conditions selected from the standardized set of clinical conditions, wherein a machine learning model assigns the first trauma activation level with an algorithm containing a plurality of relative calculable values, wherein each relative calculable value is assigned to one or more clinical conditions in the standardized set of clinical conditions;
(c) determining, by human medical review, for the individual patient which of the set of clinical conditions with which the individual patient presented;
(d) determining, by human medical review, a second trauma activation level that matches the set of clinical conditions that the individual patient presented;
(e) inputting into the machine learning model the second trauma activation level, determined by human medical review, based on the set of clinical conditions that the individual patient presented;
(f) comparing the second trauma activation level, determined by human medical review, to the first trauma activation level, assigned by the machine learning model, based on the set of clinical conditions that the individual patient presented for an individual patient identified with a plurality of clinical conditions selected from the standardized set of clinical conditions;
(g) determining, based on said comparison, whether the first trauma activation level assigned by the machine learning model corresponds to under-triage, over-triage, or an appropriate level of triage for the individual patient in a clinical environment;
(h) comparing the second trauma activation level, determined by human medical review, based on the set of clinical conditions that the individual patient presented to the ground-truth;
(i) determining, based on said comparison, whether the second trauma activation level corresponds to under-triage, over-triage, or an appropriate level of triage for the individual patient in a clinical environment; (j) adjusting one or more relative calculable values used by the machine learning model in assigning the first trauma activation level in response to the determination of whether the individual patient received under-triage, over-triage, or an appropriate level of triage for the individual patient in a clinical environment; and
(k) repeating steps (b) to (j) with a plurality of patients, each identified with a plurality of clinical conditions selected from the set of clinical conditions until the machine learning model reaches an appropriate level of triage within desired parameters.

2. The method of claim 1, wherein the desired parameters include a machine accuracy rate, wherein the machine accuracy rate is determined by:
(1) assigning a third trauma activation level, by the machine learning model, for a plurality of testing patients, each identified with a plurality of clinical conditions selected from the standardized set of clinical conditions;
(2) comparing the third trauma activation level for each testing patient to the ground-truth and determining, based on said comparison, whether the third trauma activation level assigned by the machine learning model corresponds to either under-triage, over-triage, or an appropriate level of triage for each testing patient; and
(3) calculating the machine accuracy rate for the testing patients based on the following formula
$$Machine Accuracy Rate = \frac{Number of testing patients with appropriate level of triage}{Number of total testing patients} \times 100 \% .$$

3. The method of claim 2, wherein the desired parameters further include an under-triage rate and/or an over-triage rate.

4. The method of claim 1, wherein the ground-truth is based on the Cribari matrix method (Injury Severity Score (ISS)), the Need for Trauma Intervention method (NFTI), or a combination of ISS and NFTI.

5. (canceled)

6. (canceled)

7. (canceled)

8. The method of claim 1, wherein the set of clinical conditions comprise one or more conditions selected from the group consisting of acute injury related to known or suspected child abuse, blood transfusion, hypotension, multiple rib fractures, pelvic facture, significant head injury with torso or extremity trauma, stab wound to neck, torso, head, or groin and buttock, traumatic amputation proximal to wrist or ankle, two or more suspected proximal bone fractures, Glasgow coma score of 8 or less, and gunshot wound to neck, torso, head, arm, leg, or groin and buttock.

9. The method of claim 1, wherein the set of clinical conditions comprises one or more conditions selected from the group consisting of asthma, bleeding disorders, cirrhosis or ascites, current anticoagulation, current smoker, current use of steroids, diabetes mellitus, drug use disorder, substance abuse disorder, drug abuse, functional dependency (prior to hospitalization), history of congenital anomaly, history of prematurity, history of psychiatric disorder, attention deficit disorder, personality disorder, major psychiatric disorder, history of transplant, inflammatory bowel disease. Crohn's disease, ulcerative colitis, obesity, pre-existing spinal cord injury, pre-hospital cardiac arrest, renal failure or current dialysis, and seizures.

10. (canceled)

11. (canceled)

12. A method for assigning medical resources to a subject for treatment of traumatic injury, comprising the steps of
(a) receiving, via a user interface of an application executing on one or more computer processors, a personal information profile, wherein said personal information profile comprises inputs for personal information for a plurality of types of information selected from the types of information comprising comorbidities, engineered features, prehospital interventions, injury mechanisms and numeric variables;
(b) storing, via the one or more computer processors, said personal information of said subject in a database accessible by said application, and accessible by said subject via said user interface of said application;
(c) assigning, via the one or more computer processors, a calculable value for any of a plurality of the types of information in said personal information stored in said database and a calculable value point matrix stored on a memory device accessible by the one or more computer processors, the calculable value point matrix being generated and continually updated by a machine learning module using a machine learning model trained by the method of claim 1;
(d) determining, via the one or more computer processors, a total calculable value of said types of information in said personal information for said subject via the one or more computer processors, based on evaluation in real-time, wherein when the total calculable value of said types of information in said personal information for said subject exceeds or falls below a pre-selected calculable value a predicted assignment of medical resources is generated; and
(e) notifying, via said user interface, a medical professional providing medical care to said subject about the predicted assignment of medical resources for said subject.

13. (canceled)

14. (canceled)

15. The method of claim 12, wherein said engineered features comprise one or more features selected from the group consisting of acute injury related to known or suspected child abuse, blood transfusion, hypotension, multiple rib fractures, pelvic facture, significant head injury with torso or extremity trauma, stab wound to neck, torso, head, or groin and buttock, traumatic amputation proximal to wrist or ankle, two or more suspected proximal bone fractures, Glasgow coma score of 8 or less, and gunshot wound to neck, torso, head, arm, leg, or groin and buttock.

16. The method of claim 12, wherein said comorbidities comprise one or more conditions selected from the group consisting of asthma, bleeding disorders, cirrhosis or ascites, current anticoagulation, current smoker, current use of steroids, mellitus, drug use disorder, substance abuse disorder, drug abuse, functional dependency (prior to hospitalization), history of congenital anomaly, history of prematurity, history of psychiatric disorder, attention deficit disorder, personality disorder, major psychiatric disorder, history of transplant, inflammatory bowel disease. Crohn's disease, ulcerative colitis, obesity, preexisting spinal cord injury, pre-hospital cardiac arrest, renal failure or current dialysis, and seizures.

17. The method of claim 12, wherein said prehospital interventions comprise one or more interventions selected from the group consisting of airway management, bag-valve mask ventilation, intubation, airway suction, placement of a laryngeal mark airway, placement of a supraglottic assist device, placement of oral airway, bleeding control, tourniquet, manual pressure, placement of pelvic binder, hemostatic dressing, cardiopulmonary resuscitation, electrocardiogram, intraosseous access, intravenous fluids, limb immobilization, medications, needle thoracostomy, oxygen, and spine immobilization.

18. The method of claim 12, wherein said injury mechanism comprise one or more injury mechanisms selected from the group consisting of abuse, assault, fall, gunshot wound, motor vehicle collision, pedestrian struck, sports-related injury, and stab wound.

19. The method of claim 12, wherein the numeric variables comprise one or more variables selected from the group consisting of Glasgow coma scale score, revised trauma scale, mean pre-hospital systolic blood pressure, diastolic blood pressure, heart rate, and age.

20. (canceled)

21. (canceled)

22. (canceled)

23. The method of claim 12, wherein the method further comprises the step of:
(h) receiving, via a user interface of an application executing on one or more computer processors, messages inviting said medical professional to join groups of other medical professionals using said application, wherein said groups are able to communicate amongst members of the group via the user interface.

24. The method of claim 12, further comprising the step of tracking over time, via the one or more computer processors and the total calculable values, the relative effective assignment or lack of assignment of medical resources over time with respect to trauma team activation by said medical professional.

25. The method of claim 12, further comprising the step of providing a system comprising:
(i) one or more computing devices in data communication with each other, each device having one or more computer processors, a data communication connection, and one or more tangible non-transitory computer-readable media accessible by the one or more computer processors,
(ii) a personal information database; and
(iii) a machine learning module; wherein the personal information database and the machine learning module are each stored in the one or more tangible non-transitory computer-readable media.

26. (canceled)

27. (canceled)

28. A system for assigning medical resources to a subject for treatment of traumatic injury in a subject comprising: one or more computer processors; and one or more tangible computer readable media accessible by the one or more computer processors, wherein the one or more tangible computer readable media comprise instructions that, when executed by the one or more processors, cause the one or more processors to perform:
(a) receiving, via a user interface of an application executing on one or more computer processors, a personal information profile, wherein said personal information profile comprises inputs for personal information for a plurality of types of information selected from the types of information comprising comorbidities, engineered features, prehospital interventions, injury mechanisms and numeric variables;
(b) storing, via the one or more computer processors, said personal information of said subject in a database accessible by said application, and accessible by said subject via said user interface of said application;
(c) assigning, via the one or more computer processors, a calculable value for any of a plurality of the types of information in said personal information stored in said database and a calculable value point matrix stored on a memory⁷ device accessible by the one or more computer processors, the calculable value point matrix being generated and continually updated by a machine learning module using a machine learning model trained by the method of claim 1;
(d) determining, via the one or more computer processors, a total calculable value of said types of information in said personal information for said subject via the one or more computer processors, based on evaluation in real-time, wherein when the total calculable value of said types of information in said personal information for said subject exceeds or falls below a pre-selected calculable value a predicted assignment of medical resources is generated; and
(e) notifying, via said user interface, a medical professional providing medical care to said subject about the predicted assignment of medical resources for said subject.

29. A tangible non-transitory computer readable storage medium, comprising instructions that, when executed by a computer processor, cause the processor to:
(a) receiving, via a user interface of an application executing on one or more computer processors, a personal information profile, wherein said personal information profile comprises inputs for personal information for a plurality of types of information selected from the types of information comprising comorbidities, engineered features, prehospital interventions, injury mechanisms and numeric variables;
(b) storing, via the one or more computer processors, said personal information of said subject in a database accessible by said application, and accessible by said subject via said user interface of said application;
(c) assigning, via the one or more computer processors, a calculable value for any of a plurality of the types of information in said personal information stored in said database and a calculable value point matrix stored on a memory device accessible by the one or more computer processors, the calculable value point matrix being generated and continually updated by a machine learning module using a machine learning model trained by the method of claim 1;
(d) determining, via the one or more computer processors, a total calculable value of said types of information in said personal information for said subject via the one or more computer processors, based on evaluation in real-time, wherein when the total calculable value of said types of information in said personal information for said subject exceeds or falls below a pre-selected calculable value a predicted assignment of medical resources is generated; and
(e) notifying, via said user interface, a medical professional providing medical care to said subject about the predicted assignment of medical resources for said subject.

30. The tangible non-transitory computer readable storage medium of claim 29, further comprising instructions that, when executed by a computer processor, cause the processor to:
(i) enable one or more computing devices in data communication with each other, each device having one or more computer processors, a data communication connection, and one or more tangible non-transitory computer-readable media accessible by the one or more computer processors, and
(ii) store a personal information database; and
(iii) input into a machine learning module; wherein the personal information database and the machine learning module are each stored in the one or more tangible non-transitory computer-readable media.

31. A method of training a machine learning model to predict the assignment of medical resources required to treat a patient for traumatic injury, the method comprising: (a) establishing a ground-truth, wherein the ground-truth corresponds to a standardized trauma activation level for a standardized patient with a standardized set of clinical conditions;
(b) assigning a machine trauma activation level for an individual patient identified with a plurality of clinical conditions selected from the standardized set of clinical conditions, wherein a machine learning model assigns the first trauma activation level with an algorithm containing a plurality of relative calculable values, wherein each relative calculable value is assigned to one or more clinical conditions in the standardized set of clinical conditions;
(c) comparing the machine trauma activation level to the ground-truth;
(d) determining, based on said comparison, whether the machine trauma activation level corresponds to under-triage, over-triage, or an appropriate level of triage for the individual patient;
(e) adjusting one or more relative calculable values used by the machine learning model in assigning the machine trauma activation level if the machine trauma activation level corresponds to under-triage or over-triage in step (d); and
(f) repeating steps (b) to (e) with a plurality of patients, each identified with a plurality of clinical conditions selected from the set of clinical conditions until the machine learning model reaches an appropriate level of triage within desired parameters.

32. (canceled)

33. The method of claim 31, wherein the desired parameters include a machine accuracy rate, wherein the machine accuracy rate is determined based on the following formula:
$$Machine Accuracy Rate = \frac{Number of testing patients with appropriate level of triage}{Number of total testing patients} \times 100 \% .$$

34. (canceled)
